(19) United States
(12) Patent Application Publication
(10) Pub. No.: US 2025/0191253 A1
(43) Pub. Date: Jun. 12, 2025

(54) IMAGE PROCESSING APPARATUS, IMAGING SYSTEM, IMAGE PROCESSING METHOD, AND COMPUTER-READABLE STORAGE MEDIUM

(71) Applicant: CANON KABUSHIKI KAISHA, Tokyo (JP)

(72) Inventors: Ritsuya TOMITA, Kanagawa (JP); Osamu SAGANO, Tokyo (JP)

(21) Appl. No.: 19/059,419

(22) Filed: Feb. 21, 2025

Related U.S. Application Data
(63) Continuation of application No. PCT/JP2023/030194, filed on Aug. 22, 2023.

(30) Foreign Application Priority Data
Aug. 29, 2022 (JP) 2022-136204

(51) Int. Cl. G06T 11/00; A61B 6/42; A61B 6/46; G01T 1/24; G06T 7/00

(52) U.S. Cl. CPC G06T 11/008; A61B 6/4241; A61B 6/463; G01T 1/24; G06T 7/0012; G06T 11/005; G06T 2200/24; G06T 2207/10081; G06T 2207/30004; G06T 2210/41; G06T 2210/62

(57) ABSTRACT

An image processing apparatus is provided that comprises: an obtaining unit configured to obtain an intensity image of radiation obtained by imaging a subject using the radiation, and a material decomposition image indicating a discriminated material and obtained by imaging the subject by counting photons of the radiation; and a display controlling unit configured to cause the intensity image of radiation and the material decomposition image to be juxtaposed, switched, or superimposed and displayed on a display unit.

### CROSS-REFERENCE TO RELATED APPLICATIONS

This application is a Continuation of International Patent Application No. PCT/JP2023/030194, filed Aug. 22, 2023, which claims the benefit of Japanese Patent Application No. 2022-136204, filed Aug. 29, 2022, both of which are hereby incorporated by reference herein in their entirety.

### BACKGROUND OF THE INVENTION

### Field of the Invention

The present disclosure relates to an image processing apparatus, an imaging system, an image processing method, and a computer-readable storage medium.

### Description of the Related Art

As a radiation detector used in a radiation imaging system such as an X-ray CT (Computed Tomography) apparatus, a photon counting type radiation detector is known. For example, a photon counting type X-ray detector measures the intensity of the X-rays by capturing each incident X-ray as a photon and counting the number of photons. Further, a photon counting type X-ray detector generates a charge amount corresponding to the energy of the X-ray photon when converting the X-ray photon into the electric charge, so that the energy of each X-ray photon can be measured. Therefore, the photon counting type X-ray detector can measure the energy spectrum of the X-rays.

In addition, material decomposition technology for discriminating materials contained in an object to be examined using data corresponding to a plurality of energy bands (energy bins) by utilizing the fact that the absorption characteristics of radiation are different for each material is known. By applying the material decomposition technology to the X-ray energy spectrum measured using the photon counting type X-ray detector, it is possible to obtain a material decomposition image showing the discriminated materials for the subject imaged using the radiation. Japanese Patent Application Laid-Open No. 2016-52349 discloses that an image showing the results of material decomposition obtained by the photon counting CT is displayed on a display unit.

However, in a material decomposition image using such the photon counting technology, only a specific material that has been discriminated is shown. Therefore, it may be difficult to grasp the structure of a tissue including other materials in the vicinity of the material, or there may be a site that cannot be checked. In addition, it may be desired to check the discriminated material while considering the positional relationship with a site of interest, or it may be desired to check a material decomposition image while checking a CT image, which is a conventional intensity image of radiation familiar to a physician, etc.

Accordingly, an embodiment of the present disclosure provides an image processing apparatus that can display an intensity image of radiation and a material decomposition image using the photon counting technology so as to easily compare those images.

### SUMMARY OF THE INVENTION

An image processing apparatus according to an embodiment of the present disclosure comprises: an obtaining unit configured to obtain an intensity image of radiation obtained by imaging a subject using the radiation, and a material decomposition image indicating a discriminated material and obtained by imaging the subject by counting photons of the radiation; and a display controlling unit configured to cause the intensity image of radiation and the material decomposition image to be juxtaposed, switched, or superimposed and displayed on a display unit.

Further features of the present invention will become apparent from the following description of exemplary embodiments with reference to the attached drawings.

### BRIEF DESCRIPTION OF THE DRAWINGS

FIG. 1 is a diagram for schematically illustrating an example of the configuration of a CT system according to an Embodiment 1.

FIG. 2 is a diagram for illustrating an example of data for multiple energy bands according to the Embodiment 1.

FIG. 3 is a flowchart of a series of processes according to the Embodiment 1.

FIG. 4A is a diagram for illustrating an example of an intensity image of radiation according to the Embodiment 1.

FIG. 4B is a diagram for illustrating an example of a material decomposition image of radiation according to the Embodiment 1.

FIG. 4C is a diagram for illustrating an example of a material decomposition image of radiation according to the Embodiment 1.

FIG. 5 is a diagram for illustrating an example of a display screen according to the Embodiment 1.

FIG. 6 is a diagram for illustrating another example of the display screen according to the Embodiment 1.

FIG. 7A is a diagram for illustrating another example of the display screen according to the Embodiment 1.

FIG. 7B is a diagram for illustrating another example of the display screen according to the Embodiment 1.

FIG. 7C is a diagram for illustrating another example of the display screen according to the Embodiment 1.

FIG. 8A is a diagram for illustrating another example of the display screen according to the Embodiment 1.

FIG. 8B is a diagram for illustrating another example of the display screen according to the Embodiment 1.

FIG. 9 is a diagram for schematically illustrating an example of the configuration of a CT system according to an Embodiment 2.

FIG. 10 is a diagram for illustrating an example of a machine learning model according to the Embodiment 2.

FIG. 11 is a flowchart of a series of processes according to the Embodiment 2.

FIG. 12 is a diagram for illustrating an example of a display screen according to the Embodiment 2.

FIG. 13A is a diagram for illustrating an example of a machine learning model according to a modification 5.

FIG. 13B is a diagram for illustrating an example of the machine learning model according to the modification 5.

FIG. 14A is a diagram for illustrating another example of the machine learning model according to the modification 5.

FIG. 14B is a diagram for illustrating another example of the machine learning model according to the modification 5.

### DESCRIPTION OF THE EMBODIMENTS

Preferred embodiments of the present invention will now be described in detail in accordance with the accompanying drawings. However, the dimensions, materials, shapes, relative positions of components, and the like described in the following embodiments can be freely set and may be modified depending on the configuration of an apparatus to which the present disclosure is applied or various conditions. In the drawings, the same reference numerals are used between drawings to indicate elements that are identical or functionally similar.

In the following embodiments, an imaging system using X-rays as an example of radiation is described, but other radiation may be used for an imaging system in this disclosure. Here, the term radiation may include electromagnetic radiation such as X-rays and y-rays, and particle radiation such as a-rays, B-rays, particle rays, proton rays, heavy ion rays, and meson rays. In the present disclosure, a photon such as X-ray and y-ray and particle such as B-ray and x-ray are collectively referred to as a radiation photon. In the following, an image in which a material is discriminated using data obtained by radiation imaging is referred to as a material decomposition image. On the other hand, a fluoroscopic image such as a CT image or radiation image, etc. obtained by a CT system, radiation imaging apparatus, etc., which is not subjected to material decomposition, is referred to as an intensity image of radiation. In the following embodiments, a still image will be described as an image to which this disclosure applies, but the image to which this disclosure applies may be a moving image.

In the following embodiments, an imaging system using CT will be described as an example of a radiation imaging system, but an imaging system in this disclosure is not limited thereto. For example, an imaging system of DR (Digital Radiography) using an FPD (Flat Panel Detector), or an imaging system of PET (Positron Emission Tomography) may be used. Further, an imaging system of SPECT (Signal Photon Emission Computed Tomography) may be used. The radiation imaging system described above may be used as a radiation diagnostic apparatus.

Furthermore, in the following embodiments, an imaging system for imaging a human body as a subject in the medical field will be described. However, the present disclosure may be applied to an imaging system for imaging a subject for non-destructive inspection in the industrial field.

### Embodiment 1

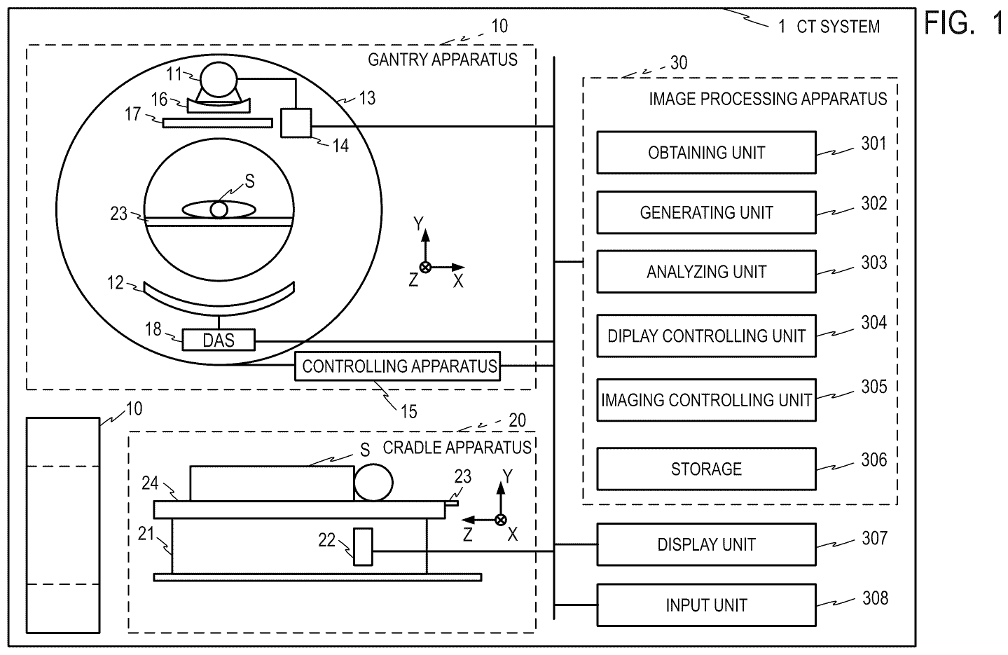

Hereinafter, referring to FIG. 1 to FIG. 8B, a CT system 1 using an X-ray and an image processing method according to an Embodiment 1 will be described. The CT system 1 according to the Embodiment 1 can perform the photon counting CT. In the photon counting CT, a CT image with a high SN ratio can be reconstructed by counting radiation transmitted through an object to be examined using a photon-counting type radiation detector capable of counting photon in radiation.

FIG. 1 is a diagram for schematically illustrating an example of the configuration of the CT system 1 using the X-ray according to the Embodiment 1. As shown in FIG. 1, the CT system 1 according to the Embodiment 1 includes a gantry apparatus 10, a cradle apparatus 20, and an image processing apparatus 30.

In FIG. 1, the rotational axis of a rotating frame 13 in the non-tilt state or the longitudinal direction of a top plate 23 of the cradle apparatus 20 is defined as the Z-axis direction. The X-axis direction is an axial direction perpendicular to the Z-axis direction and horizontal to a floor surface. The Y-axis direction is an axial direction perpendicular to the Z-axis direction and vertical to the floor surface. FIG. 1 is a diagram for showing the gantry apparatus 10 from a plurality of directions for illustration, and shows a case where the CT system 1 has one gantry apparatus 10.

The gantry apparatus 10 is provided with an X-ray tube 11, an X-ray detector 12, a rotating frame 13, an X-ray high-voltage apparatus 14, a controlling apparatus 15, a wedge 16, a collimator 17, and a DAS (Data Acquisition System) 18.

The X-ray tube 11 is a vacuum tube having a cathode (filament) that generates thermal electrons and an anode (target) that generates X-rays based on the collision of the thermal electrons. The X-ray tube 11 generates the X-rays irradiated to the object to be examined S by applying a high voltage from the X-ray high-voltage apparatus 14 to irradiate thermal electrons from the cathode to the anode. For example, the X-ray tube 11 includes a rotating anode type X-ray tube that generates the X-rays by irradiating the thermal electrons to a rotating anode.

The rotating frame 13 is an annular frame which supports the X-ray tube 11 and the X-ray detector 12 so as to face each other and rotates the X-ray tube 11 and the X-ray detector 12 according to the controlling apparatus 15. For example, the rotating frame 13 may be a cast made of aluminum. In addition to the X-ray tube 11 and the X-ray detector 12, the rotating frame 13 may further support the X-ray high voltage apparatus 14, the wedge 16, the collimator 17, the DAS 18, etc. The rotating frame 13 may further support various configuration not shown.

The wedge 16 is a filter for adjusting the X-ray dose irradiated from the X-ray tube 11. Specifically, the wedge 16 is a filter for transmitting and attenuating the X-rays irradiated from the X-ray tube 11 so that the X-rays irradiated from the X-ray tube 11 to the object to be examined S have a predetermined distribution. For example, the wedge 16 may be a wedge filter or a bow-tie filter, or a filter made of aluminum or the like so as to have a predetermined target angle and a predetermined thickness.

The collimator 17 is a lead plate or the like for narrowing the irradiation range of the X-rays transmitted through the wedge 16, and a slit is formed by combining a plurality of lead plates or the like. The collimator 17 is sometimes called an X-ray diaphragm. FIG. 1 shows a case where the wedge 16 is disposed between the X-ray tube 11 and the collimator 17, but the collimator 17 may be disposed between the X-ray tube 11 and the wedge 16. In this case, the wedge 16 transmits and attenuates X-rays irradiated from the X-ray tube 11 and of which the irradiation range is limited by the collimator 17.

The X-ray high-voltage apparatus 14 has an electric circuit such as a transformer and a rectifier, and is provided with a high-voltage generator that generates a high voltage applied to the X-ray tube 11, and an X-ray controlling apparatus that controls an output voltage corresponding to the X-rays generated by the X-ray tube 11. The high-voltage generator may be a transformer type or an inverter type. The X-ray high-voltage apparatus 14 may be provided in the rotating frame 13 or in a fixed frame (not shown).

The controlling apparatus 15 has a processing circuit including a CPU (Central Processing Unit) and a drive mechanism such as a motor and an actuator. The controlling apparatus 15 controls the operation of the gantry apparatus 10 and the cradle apparatus 20 in response to an input signal from an input unit 308. For example, the controlling apparatus 15 controls the rotation of the frame 13, the tilt of the gantry apparatus 10, and the operation of the cradle apparatus 20 and the top plate 23. In an example, the controlling apparatus 15 rotates the frame 13 about an axis parallel to the X-axis direction according to the input tilt angle information as the control for tilting the gantry apparatus 10. The controlling apparatus 15 may be provided in the gantry apparatus 10 or the image processing apparatus 30.

Each time the X-ray photon is incident, the X-ray detector 12 outputs a signal capable of measuring the energy value of the X-ray photon. The X-ray photon is, for example, an X-ray photon irradiated from the X-ray tube 11 and transmitted through the object to be examined S. The X-ray detector 12 has a plurality of detection elements which output an electric signal (analog signal) of one pulse each time the X-ray photon is incident. Therefore, by counting the number of the electric signals (pulses) output from each detection element, the number of the X-ray photons incident on each detection element can be counted. Further, by performing arithmetic processing on this signal, the energy value of the X-ray photon which caused the output of the signal can be measured.

The above detection element is, for example, a semiconductor detection element such as CdTe (Cadmium Telluride) or CdZnTe (Cadmium Zinc Telluride) with electrodes arranged on it. That is, the X-ray detector 12 is a direct conversion type detector that directly converts the incident X-ray photon into an electric signal. The X-ray detector 12 is not limited to the direct conversion type detector, but may be an indirect conversion type detector that, for example, converts the X-ray photon into visible light using a scintillator, etc., and converts the visible light into an electric signal using a photosensor, etc.

The X-ray detector 12 is provided with the above detection elements and a plurality of ASICs (Application Specific Integrated Circuits) that are connected to the detection elements and count the X-ray photon detected by the detection elements. The ASIC counts the number of X-ray photons incident on the detection element by distinguishing the individual charges output by the detection element. The ASIC also measures the energy of the counted X-ray photons by performing arithmetic processing based on the magnitude of the individual charges. Furthermore, the ASIC outputs the count result of the X-ray photons to the DAS 18 as digital data.

The DAS 18 generates detection data based on the result of the count processing input from the X-ray detector 12. The detection data is, for example, a sinogram. The sinogram is data in which the results of counting processing of the radiation incident on each detection element at each position of the X-ray tube 11 are arranged. The sinogram is data in which the results of counting processing are arranged in a two-dimensional rectangular coordinate system with the view direction and the channel direction as axes. The DAS 18 generates a sinogram, for example, in column units in the slice direction of the X-ray detector 12. The DAS 18 transfers the generated detection data to the image processing apparatus 30. The DAS 18 can be implemented by a processor such as a CPU, for example.

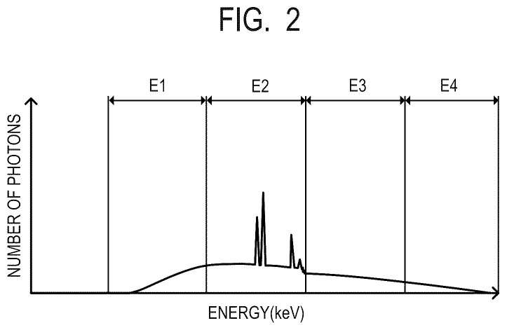

Here, the result of the counting process is data in which the number of photons of X-rays is allocated for each energy bin (energy bands E1 to E4) as shown in FIG. 2. For example, the DAS 18 counts the photons (X-ray photons) derived from the X-rays irradiated from the X-ray tube 11 and transmitted through the object to be examined S, and discriminates the energy of the counted X-ray photons as the result of the counting process. FIG. 2 shows an example of a plurality of energy bands, but the number and width of the energy bands to be discriminated are not limited thereto, and may be set according to a desired configuration.

The data generated by the DAS 18 is transmitted by optical communication from a transmitter having a light emitting diode (LED) provided in the rotating frame 13 to a receiver having a photodiode provided in the non-rotating portion of the gantry apparatus 10, and transferred to the image processing apparatus 30. The non-rotating portion may be, for example, a fixed frame (not shown) that rotatably supports the rotating frame 13. The data transmission method from the rotating frame 13 to the non-rotating portion of the gantry apparatus 10 is not limited to the optical communication, and any non-contact data transmission method or a contact data transmission method may be adopted.

The cradle apparatus 20 is an apparatus for mounting and moving the object to be examined S as an imaging-target, and the cradle apparatus 20 is provided with a base 21, a cradle driving apparatus 22, a top plate 23, and a support frame 24. The cradle apparatus 21 is a casing for vertically movably supporting the support frame 24. The cradle driving apparatus 22 is a driving mechanism for moving the top plate 23 on which the object to be examined S is mounted in the longitudinal direction of the top plate 23, and includes a motor, an actuator, and the like. The top plate 23 provided on the upper surface of the support frame 24 is a plate on which the object to be examined S is mounted. The cradle driving apparatus 22 may move the support frame 24 in the longitudinal direction of the top plate 23 as well as the top plate 23.

The image processing apparatus 30 is provided with an obtaining unit 301, a generating unit 302, an analyzing unit 303, a display controlling unit 304, an imaging controlling unit 305, and a storage 306. A display unit 307, an input unit 308, the gantry apparatus 10, and the cradle apparatus 20 are communicably connected to the image processing apparatus 30. The image processing apparatus 30 and gantry apparatus 10 are described separately in the Embodiment 1, however the gantry apparatus 10 may include the image processing apparatus 30 or a part of the components of the image processing apparatus 30.

Note that the image processing apparatus 30 may be configured by a computer provided with a processor and memory. Each component of the image processing apparatus 30 other than the storage 306 is functionally configured by using, for example, one or more processors such as a CPU and a program read from the storage 306. The processor may be, for example, a Micro Processing Unit (MPU), a Graphical Processing Unit (GPU), a Field-Programmable Gate Array (FPGA), or the like. Each component of the image processing apparatus 30 other than the storage 306 may be configured by an integrated circuit such as an ASIC that performs a specific function. Further, as an internal configuration of the image processing apparatus 30, it is also possible to include a graphic controlling unit such as a GPU, a communication unit such as a network card, and an input/output controlling unit such as a keyboard, display, or touch panel.

The obtaining unit 301 can obtain data generated by the DAS 18 and various operations, patient information, and the like input from an operator via the input unit 308. Furthermore, the obtaining unit 301 may obtain data obtained by imaging the object to be examined S, a CT image of the object to be examined S, a material decomposition image of the object to be examined S, the patient information, and the like from an image processing apparatus or a storage apparatus (not shown) connected to the image processing apparatus 30 via any network. The network may include, for example, a LAN (Local Area Network), an intranet, the Internet, or the like.

The generating unit 302 performs pre-processing such as logarithmic transformation processing, offset correction processing, sensitivity correction processing between channels, and beam hardening correction on the data output from the DAS 18 to generate projection data. The generating unit 302 also performs reconstruction processing using the filtered back projection method, the successive approximation reconstruction method, and the like on the generated projection data to generate a CT image. The generating unit 302 stores the reconstructed CT image in the storage 306.

Here, the projection data generated from the count result obtained by the photon counting CT includes information on the energy of X-rays attenuated by transmitting through the object to be examined S. Therefore, the generating unit 302 can reconstruct the CT images of a specific energy band. The generating unit 302 can reconstruct the CT images of each of a plurality of energy bands. The CT images (of all energy bands) reconstructed without dividing them by energy bands correspond to an intensity image of radiation.

Furthermore, the generating unit 302 can generate a plurality of color-coded CT images by assigning color tones corresponding to energy bands, for example, to the CT images of each energy band. Furthermore, the generating unit 302 can generate an image obtained by superimposing a plurality of color-coded CT images corresponding to the energy bands.

Furthermore, the generating unit 302 can generate a material decomposition image that can identify the material by using, for example, the K-edge inherent to the material. The method for generating a material decomposition image is not limited to the method using the K-edge, and any known method may be used. Regarding the material decomposition image, the generating unit 302 can generate a material decomposition image that is color-coded according to the material and an image obtained by superimposing a plurality of color-coded material decomposition images, similarly to the CT image that is color-coded according to the energy bands described above. The generating unit 302 can also generate, for example, a monochromatic X-ray image, a density image, an effective atomic number image, and the like.

In order to reconstruct the CT image, projection data of a circumference of the object to be examined S of 360° is required, and even in the half-scan method, projection data of 180°+a fan angle is required. The Embodiment 1 can be applied to any of the reconstruction methods. Hereinafter, for the purpose of simplifying the explanation, a reconstruction method (full scan reconstruction) in which projection data of a circumference of the object to be examined S of 360° is used for reconstruction will be used.

Further, the generating unit 302 can convert, by a known method, the generated CT image into a tomographic image of any cross section, a three-dimensional image by rendering processing, or the like based on input from the operator via the input unit 308. The generating unit 302 stores the generated CT image, the generated material decomposition image, the converted tomographic image, or the converted three-dimensional image in storage 306.

The analyzing unit 303 performs desired analysis processing using various images generated by the generating unit 302. The analyzing unit 303 performs image processing on, for example, the CT image or the material decomposition image generated by the generating unit 302, and obtain an analysis result such as the size of an abnormal site of the object to be examined S and the density of material contained in the tissue. The analyzing unit 303 may perform analysis processing using projection data before generating an image. The analyzing unit 303 stores the generated analysis results in the storage 306.

The display controlling unit 304 causes the display unit 307 to display the patient information, various types of images, the analysis result, and information related to the various types of images stored in the storage 306. In particular, the display controlling unit 304 according to the Embodiment 1 causes the display unit 307 to display a CT image as an intensity image of radiation and a material decomposition image generated using photon counting technology in a manner that makes it easy to compare them. For example, the display controlling unit 304 causes the CT image and the material decomposition image to be displayed side by side, switched, or superimposed.

The imaging controlling unit 305 controls the CT scan performed in the gantry apparatus 10. For example, the imaging controlling unit 305 controls the operation of the X-ray high voltage apparatus 14, the X-ray detector 12, the controlling apparatus 15, the DAS 18, and the cradle driving apparatus 22, thereby controlling the collection processing of the count results in the gantry apparatus 10. For example, the imaging controlling unit 305 controls the collection processing of projection data in imaging for a positioning-image (scanogram image) and main imaging (scan) for collecting images used for observation, respectively.

The storage 306 is realized by, for example, a semiconductor memory element such as a RAM (Random Access Memory), a flash memory, a hard disk, an optical disk, etc. The storage 306 stores, for example, the patient information, the projection data, the various images such as the CT image and the material decomposition image, the analysis results, the information related to the various images, etc. Also, for example, the storage 306 can store programs for realizing the functions of the above-described components. The storage 306 may be realized by a group of servers (cloud) connected to the CT system 1 via a network.

The display unit 307 displays various types of information. For example, the display unit 307 may display the various images generated by the generating unit 302, or display a GUI (Graphical User Interface) for receiving various operations from an operator. The display unit 307 may be any display such as a liquid crystal display, an organic EL display, or a CRT (Cathode Ray Tube) display. The display unit 307 may be a desktop type or a tablet terminal capable of wireless communication with the image processing apparatus 30.

The input unit 308 receives various input operations from the operator, converts the received input operations into electrical signals, and outputs them to the image processing apparatus 30. Also, for example, the input unit 308 receives input operations such as reconstruction conditions for reconstructing a CT image or image processing conditions for generating a post-processing image from the CT image from the operator.

For example, the input unit 308 may be realized by a mouse, a keyboard, a trackball, a switch, a button, a joystick, a touch pad capable of performing input operations by touching an operation surface, or a touch screen integrated with a display screen and a touch pad. The input unit 308 may be realized by a non-contact input circuit using an optical sensor, a voice input circuit, or the like. The input unit 308 may be provided in the gantry apparatus 10. The input unit 308 may be configured by a tablet terminal or the like capable of wireless communication with the image processing apparatus 30. Furthermore, the input unit 308 is not limited to a device having a physical operation component such as a mouse or keyboard. For example, the input unit 308 includes an electrical signal processing circuit that receives an electrical signal corresponding to an input operation from an external input device provided separately from the image processing apparatus 30 and outputs the electrical signal to the image processing apparatus 30.

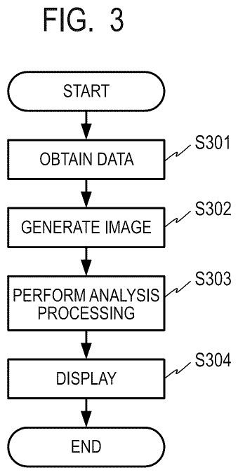

Next, a series of processes including image processing according to the Embodiment 1 will be described with reference to FIG. 3 to FIG. 8B. FIG. 3 is a flowchart of a series of processes according to the Embodiment 1. When the process according to the Embodiment 1 is started in response to an instruction or the like from an operator, the process proceeds to step S301.

In step S301, the obtaining unit 301 obtains data obtained by imaging the object to be examined S using the gantry apparatus 10 based on imaging-condition or the like input by the operator. Here, the obtained data includes a count result obtained by the photon counting CT. Further, the obtaining unit 301 may obtain data obtained by imaging the object to be examined S from an image processing apparatus or a storage device (not shown) via any network.

Next, in step S302, the generating unit 302 generates a CT image based on the obtained data. The generating unit 302 generates a material decomposition image based on the count result included in the obtained data. As described above, the method of generating the material decomposition image may be a method using the K-edge or any other known method.

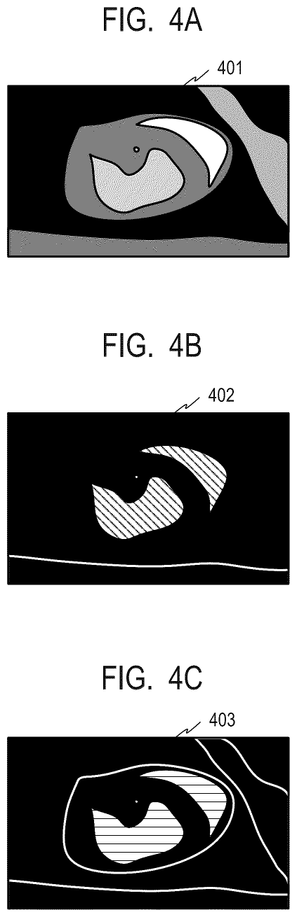

FIG. 4A to FIG. 4C show schematic examples of CT images and material decomposition images for one cross section of the object to be examined S. FIG. 4A shows a CT image 401. The CT image 401 is an intensity image of radiation corresponding to a conventional CT image, and is an image in which a pixel value is a CT value obtained using data of all energy bands included in the projection data. FIG. 4B shows a material decomposition image 402 for iodine obtained using the projection data. FIG. 4C shows a material decomposition image 403 for gadolinium obtained using the projection data.

The material decomposition images are not limited to those relating to iodine and gadolinium, and the generating unit 302 may generate material decomposition images discriminating calcium, bone, soft tissue, and other materials. In FIG. 4B and FIG. 4C, a predetermined pattern of hatching is used in order to make it easy to understand the difference between material decomposition images, but in practice, values to which color tones corresponding to the materials are assigned may be used as each pixel value.

The generating unit 302 can generate an image of a cross section corresponding to an instruction of the operator or a pre-setting with respect to a three-dimensional CT image or the material decomposition image generated using projection data. In the following description, for the sake of simplicity, the CT image and the material decomposition image will be described as images of a cross section. In addition, the generating unit 302 may generate an image of each energy band, a monochromatic X-ray image, or the like, as described above. Note that the pre-setting may include at least one of setting preset for each imaging-condition including imaged site, or setting preset for each imaging-mode according to disease, etc.

Next, in step S303, the analyzing unit 303 performs analysis processing using the various images generated in step S302. The analysis processing may include detection of an abnormal site, calculation of the density of a predetermined material, or the like. However, the analysis processing is not limited to these, and any analysis processing required in the medical field or the industrial field may be performed according to the desired configuration. Noted that the analyzing unit 303 may perform the analysis processing using projection data before generating an image. Moreover, the analysis processing may be omitted according to the instruction of the operator or the pre-setting.

In step S304, the display controlling unit 304 causes the display unit 307 to display the CT image and the material decomposition image in a display mode in which they can be easily compared. As described above, by using the photon counting technology, it is possible to generate a material decomposition image in which a desired material is distinguished. However, in the material decomposition image 402,403 shown in FIG. 4B and FIG. 4C, it is possible to grasp the tissue containing the material to be distinguished, but it is difficult to grasp the surrounding tissue which does not contain the material to be distinguished. Therefore, it may be difficult to grasp the relationship between the respective tissues. In addition, it may be desired to check the material decomposition image while checking the CT image, which is a familiar conventional intensity image of radiation.

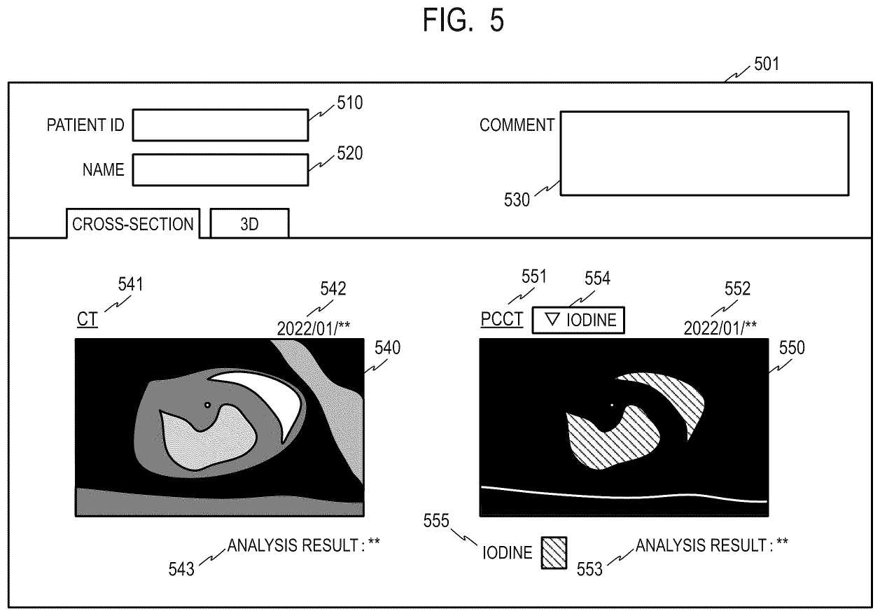

Therefore, the display controlling unit 304 according to the Embodiment 1 causes the CT image and the material decomposition image to be juxtaposed, switched, or superimposed and displayed so that they can be easily compared with each other. Now, with reference to FIG. 5 to FIG. 8B, display screens which the display controlling unit 304 according to the Embodiment 1 causes the display unit 307 to display will be described.

FIG. 5 shows a display screen 501 which displays a CT image 540 and a material decomposition image 550 side by side as an example of the display screen according to the Embodiment 1. The display screen 501 shows an example of the material decomposition image 550 in which iodine is discriminated. The display screen 501 shows the patient ID 510, the name of the patient 520, the comment 530 about the patient and the image, the CT image 540, and the material decomposition image 550. Further, kinds 541, 551 of the images, imaging dates 542, 552 of the images, and analysis result 543, 553 are shown around the CT image 540 and the material decomposition image 550. Furthermore, a kind 554 of the material to be discriminated and a display example 555 of the color tone corresponding to the material are shown around the material decomposition image 550.

For the patient ID 510, the name 520, and the comment 530, the display controlling unit 304 can read and display the information stored in the storage 306. The patient ID 510, the name 520, and the comment 530 may be stored in the storage 306 in association with the CT image 540 and the material decomposition image 550. As the comment 530, for example, the name of the disease, the examined site, the presence or absence of imaging failure in the image, the reason for the imaging failure, and the like may be displayed.

The patient ID 510, the name 520, and the comment 530 may be additionally input by the operator via the input unit 308, and the display controlling unit 304 may store the input information in the storage 306 in association with the CT image 540 and the material decomposition image 550. In addition, the presence or absence and the reason of imaging failure may be received by providing a separate selection button or input frame for the input from the operator.

The kinds 541, 551 of the images indicate the kinds of the displayed CT image 540 and the displayed material decomposition image 550, respectively. In the example shown in FIG. 5, the kind 541 of the image indicates a CT image, and the kind 551 of the image indicates a PCCT image (material decomposition image based on photon count).

The imaging dates 542, 552 of the images indicates the imaging dates of the displayed CT image 540 and the displayed material decomposition image 550. It should be noted that the CT image 540 and the material decomposition image 550 may be displayed for the comparison for the purpose of, for example, making it easier to grasp the relationship between tissues, and they may not be images obtained in the same imaging. Therefore, by display the imaging dates 542, 552 of the images, the operator can understand when the respective images were taken, and the difference in the tissue structure in the images can be understood as due to the change with time.

The analysis result 543, 553 shows the result of the analysis processing performed using each image. The analysis result need not be displayed for each image, and may be displayed by providing a single display frame. Further, the analysis result need not be a value, and for example, an area of an abnormal site or the like may be detected and displayed as a result of the analysis. In this case, the region corresponding to the detected abnormal site may be emphasized and displayed in the corresponding image so that the region corresponding to the detected abnormal can be easily grasped, according to the operator's instruction such as ON/OFF of a button (not shown).

The kind 554 of the material indicates the material to be discriminated in the material decomposition image 550, and iodine is shown as an example on the display screen 501. With respect to the kind 554 of the material, the display controlling unit 304 can display the material to be discriminated so that the operator can select, for example, options which the operator can select can be displayed. Further, the display controlling unit 304 can display a material decomposition image corresponding to the material selected according to the instruction of the operator as the material decomposition image 550.

In the medical field, a kind of the material may include, for example, iodine, gadolinium, calcium, bone, soft tissue, any metal, etc., and in the industrial field, it may include, for example, solder, silicon, etc. In addition, the kind of the material may include other materials according to the desired configuration. As the kind of the material, a plurality of materials may be selected, for example, iodine, gadolinium, etc. In this case, an image in which a material decomposition image of iodine and a material decomposition image of gadolinium are superimposed on each other can be displayed as the material decomposition image 550.

The display example 555 of the color tone or the like corresponding to the material exemplifies the color tone or the like corresponding to the material displayed in the material decomposition image 550. The display example 555 may include color tones, display patterns, etc. The display example 555 of the color tone or the like corresponding to the material can be useful for an operator to identify the discriminated material in the material decomposition image 550 and an image on which the material decomposition image 550 is superimposed.

Since the CT image 540 and the material decomposition image 550 are displayed side by side on the display screen 501, the operator can easily compare these images. Therefore, it is easier for the operator to grasp the relationship between the tissue containing the material to be discriminated and the tissue not containing the material to be discriminated than when the operator observes the material decomposition image 550 alone, and the operator can perform the observation more efficiently.

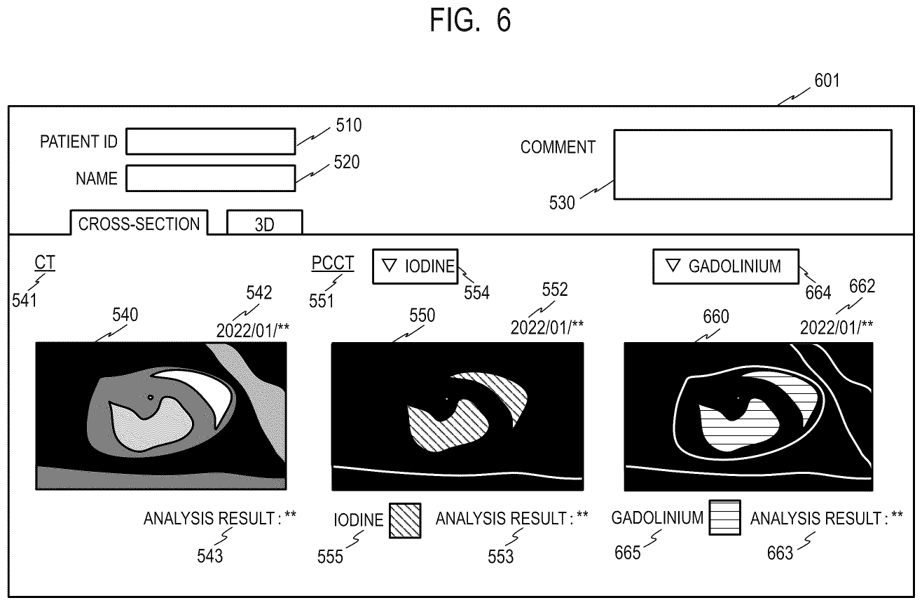

In the example shown in FIG. 5, the CT image 540 and one material decomposition image 550 are displayed side by side. On the other hand, the CT image and a plurality of kinds of material decomposition images may be displayed side by side. FIG. 6 shows a display screen 601 which displays the CT image 540 and two material decomposition images 550, 660 side by side as an example of the display screen according to the Embodiment 1. In the example shown in FIG. 6, compared with the example shown in FIG. 5, the material decomposition image 660, an imaging date 662 of the image, an analysis result 663, a kind 664 of the material, and a display example 665 of the color tone and the like corresponding to the material are shown.

The material decomposition image 660 is an example of a material decomposition image in which gadolinium is discriminated. Each of the imaging date 662 of the image, the analysis result 663, the kind 664 of the material, and the display example 665 of color tones and the like corresponding to the material corresponds to the material decomposition image 660. The imaging date 662 of the image, the analysis result 663, the kind 664 of the material, and the display example 665 of color tones and the like corresponding to the material may be similar to the imaging date 552 of the image, the analysis result 553, the kind 554 of the material, and the display example 555 of color tones and the like corresponding to the material.

Since the CT image 540 and the material decomposition image 550, 660 are displayed side by side on such the display screen 601, the operator can easily compare the CT image and the material decomposition images. In addition, because a plurality of material decomposition images 550, 660 for different kind materials are displayed side by side, the operator can easily compare the tissues containing the materials for different materials. Therefore, the operator can easily grasp the relationship between the tissues containing different materials, and can perform the observation more efficiently.

In a case where the CT image 540 and the plurality of material decomposition images 550, 660 are displayed side by side, the plurality of material decomposition images 550, 660 may be arranged so as to be adjacent to the CT image 540 on the upside and downside, and/or on the left and right sides. For example, the material decomposition image 550 may be displayed on the left side of the CT image 540, and the material decomposition image 660 may be displayed on the right side of the CT image 540. In this case, the operator can easily compare the respective material decomposition images 550, 660 and the CT image 540, and can perform observation more efficiently.

In the examples shown in FIG. 5 and FIG. 6, the material decomposition images in which one material is discriminated are displayed as the material decomposition images. On the other hand, a material decomposition image obtained by superimposing a plurality of material decomposition images in which different kinds of materials are discriminated may be displayed side by side with a CT image.

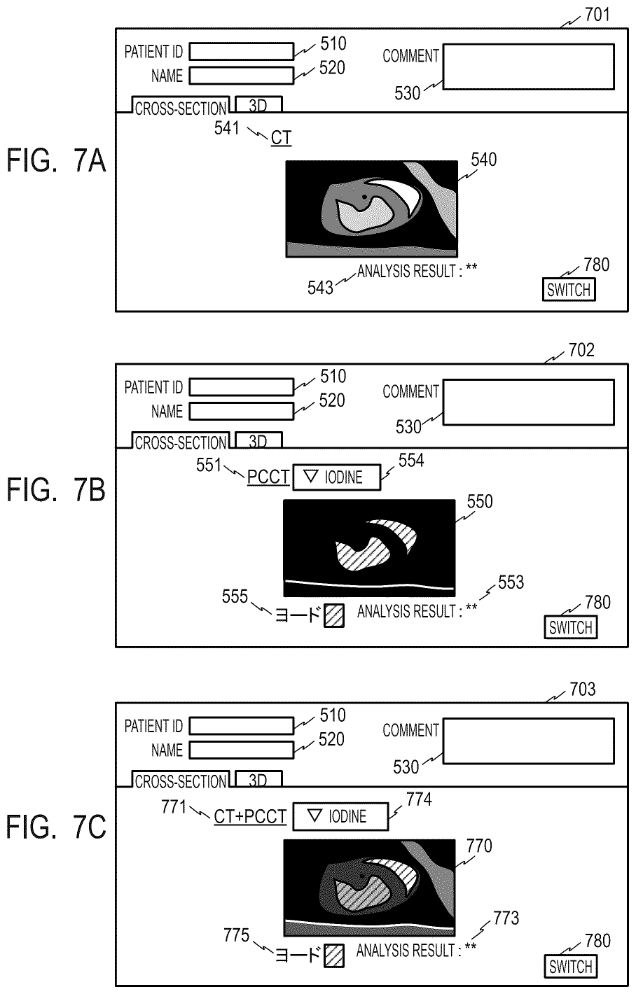

FIG. 7A and FIG. 7B show a display screen 701, 702 in which a CT image 540 and a material decomposition image 550 are switched and displayed, as an example of the display screen according to the Embodiment 1. The same configuration as in FIG. 5 is denoted by the same reference numerals and a detailed description thereof is omitted. In FIG. 7A and FIG. 7B, the imaging date of the image is omitted to simplify the description, but the image date of the image may be indicated on each display screen.

In the display screen 701 shown in FIG. 7A, only the CT image 540 is shown as a display image and a switching button 780 is shown. If the switching button 780 is operated according to the operator's instruction, the display screen 701 is switched to the display screen 702 shown in FIG. 7B. In the display screen 702, only the material decomposition image 550 is shown as display image. Further, if the switching button 780 is operated according to the operator's instruction, the display screen 702 is switched to the display screen 701 shown in FIG. 7A.

Therefore, since the CT image 540 and the material decomposition image 550 are switched and displayed on the display screens 701, 702, the operator can easily compare these images. Therefore, it is easier for the operator to grasp the relationship between the tissue containing the target material and the tissue not containing the target material than when the material decomposition image 550 is observed alone, and the operator can perform the observation more efficiently.

In addition, in the display screen 701 and display screen 702, the positions and sizes of the displays of the CT image 540 and the material decomposition image 550 are matched, so that the operator can observe these images in a manner that makes it easier to compare them. Furthermore, the material decomposition image to be switched from the CT image and displayed is not limited to one, but may be a plurality of material decomposition images discriminating different kinds of materials. In this case, by operating the switch button 780, the plurality of material decomposition images can be displayed to be switched in a preset order. In addition, as the material decomposition image, a material decomposition image in which a plurality of material decomposition images discriminating different kinds of materials are superimposed may be displayed.

In FIG. 7A and FIG. 7B, the display screen 701 for displaying the CT image 540 and the display screen 702 for displaying the material decomposition image 550 are switched according to the operation of the switch button 780. However, the display controlling unit 304 may, for example, provide a slider (not shown) and cause display unit 307 to display an image obtained by blending (superimposing) the CT image 540 and the material decomposition image 550 with each other in blend ratio according to the operation of the slider. Here, the blend ratio may be a ratio of transparency having such a relationship in which one of transparency of the images to be superimposed on each other is raised and the other is lowered. In this case, the display controlling unit 304 can set the blend ratio between the CT image 540 and the material decomposition image 550 according to the operator's instruction, superimpose the CT image 540 and the material decomposition image 550 on each other in the set blend ratio, and cause the display unit 307 to display the blended image. In addition, the display controlling unit 304 may set transparency for either image according to the operation of the slider, superimpose the image on the other image, and cause the display unit 307 to display the superimposed image. Even in these cases, the operator can easily compare the CT image 540 and the material decomposition image 550.

FIG. 7C shows a display screen 703 displaying an image in which the material decomposition image 550 is superimposed on the CT image 540. The same configuration as in FIG. 5 is denoted by the same reference numerals, and a detailed description thereof is omitted. In FIG. 7C, the imaging date of the image is omitted for simplicity of explanation, but the imaging date of the image may be indicated on the display screen.

The display screen 703 shown in FIG. 7C shows a superimposed image 770 in which the material decomposition image 550 is superimposed on the CT image 540. A kind 771 of the image, an analysis result 773, a kind 774 of the material, and a display example 775 of the color tone and the like corresponding to the material correspond to the superimposed image 770. The analysis result 773, the kind 774 of the material, and the display example 775 of the color tone and the like corresponding to the material may be similar to the analysis result 553, the kind 554 of the material, and the display example 555 of the color tone and the like corresponding to the material. The kind 771 of the image is CT+PCCT so as to indicate that the material decomposition image is superimposed on the CT image. The analysis result 773 may correspond to the CT image 540 and/or the material decomposition image 550.

The superimposed image 770 may be a superimposed image in which the CT image 540 is superimposed on the material decomposition image 550. The material decomposition image is not limited to one kind of the material decomposition image, but may also be a material decomposition image obtained by superimposing a plurality of material decomposition images in which different kinds of materials are discriminated respectively.

The display controlling unit 304 may switch the display screen 701 or the display screen 702 to the display screen 703 when the switching button 780 shown in FIG. 7A and FIG. 7B is operated. In this case, since the CT image 540 and the material decomposition image 550 are superimposed and displayed, the operator can easily compare these images. Further, since the CT image 540 or the material decomposition image 550 which are not superimposed on each other and the superimposed image 770 are switched and displayed, the operator can easily compare these images.

Regarding the superimposed image 770, the material decomposition image 550 for which the transparency according to the operator's instruction or a predetermined setting is set may be superimposed on the CT image 540, and the CT image 540 for which the transparency is set similarly may be superimposed on the material decomposition image 550.

Also, in the example in which the CT image and the material decomposition image are displayed side by side shown in FIGS. 5 and 6, the switching button 780 may be provided, and the respective images and the superimposed image may be switched and displayed according to the operation of the switching button 780. In this case, the switching button 780 may be provided for each of the CT image and the material decomposition image. For example, when the switching button 780 corresponding to the CT image is operated, the display of the CT image can be switched to the display of the superimposed image in which the material decomposition image is superimposed on the CT image. Similarly, when the switch button 780 corresponding to the material decomposition image is operated, the display of the material decomposition image can be switched to the display of the superimposed image in which the CT image is superimposed on the material decomposition image. Even in this case, the operator can easily compare these images.

In a case where the CT image and the plurality of material decomposition images are displayed side by side, options may be provided so as to select the material decomposition image to be superimposed on the CT image according to the operator's instruction. In a case where the CT image is superimposed on the plurality of material decomposition images, the plurality of material decomposition images can be collectively switched to the superimposed image according to the operation of the switch button 780. A switch button for switching between the material decomposition image and the superimposed image may be provided for each of the plurality of material decomposition images.

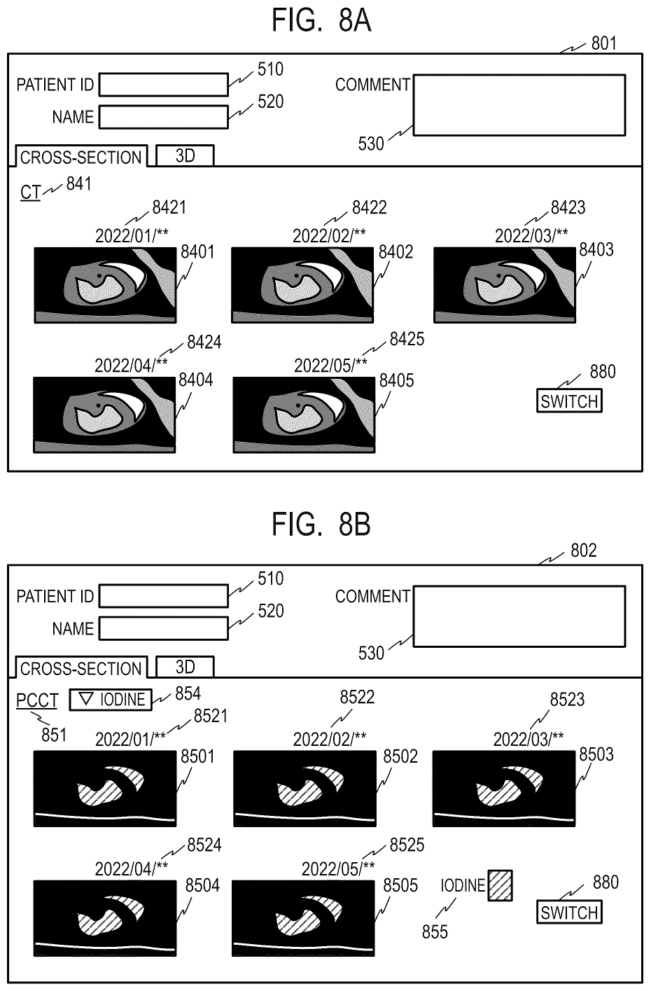

FIG. 8A and FIG. 8B show, as an example of the display screen according to the Embodiment 1, display screens 801, 802 for follow-up observation in which a plurality of CT images and a plurality of material decomposition images are switched and displayed. The same configuration as that of FIG. 5 is denoted by the same reference numerals and detailed description is omitted. The analysis results are omitted in FIG. 8A and FIG. 8B for the sake of simplicity of explanation, however the analysis results may be shown on each display screen.

The display screen 801 shown in FIG. 8A shows CT images 8401, 8402, 8403, 8404, 8405 obtained at different times. In addition, the display screen 801 shows a kind 841 of the images, imaging dates 8421, 8422, 8423, 8424, 8425 corresponding to each image, and a switching button 880. When the switching button 880 is operated in response to the operator's instruction, the display screen 801 switches to the display screen 802 shown in FIG. 8B.

The display screen 802 shows material decomposition images 8501, 8502, 8503, 8504, 8505 corresponding to the CT images 8401, 8402, 8403, 8404, 8405 obtained at different times. In addition, the display screen 802 shows a kind 851 of images, imaging dates 8521, 8522, 8523, 8524, 8525 corresponding to each image, a kind 854 of material, a display example 855 of color tones and the like corresponding to the material, and a switching button 880. The kinds 841, 851 of the images, the kind 854 of the material, and the display example 855 of color tones and the like corresponding to the material may be similar to the kinds 541, 551 of the images, the kind 554 of material, and the display example 555 of color tones and the like corresponding to the material in FIG. 5. Further, when the switching button 880 is operated according to the operator's instruction, the display screen 802 switches to the display screen 801 shown in FIG. 8A.

Therefore, on the display screen 801,802, the plurality of CT images 8401, 8402, 8403, 8404, 8405 and the plurality of material decomposition images 8501, 8502, 8503, 8504, 8505 are collectively switched and displayed. Thus, the operator can easily compare these images. The switching button 880 may be provided for each image.

Note that the CT images 8401, 8402, 8403, 8404, 8405 and superimposed images in which the corresponding material decomposition images 8501, 8502, 8503, 8504, 8505 are superimposed on the CT images may be switched and displayed. Similarly, the material decomposition images 8501, 8502, 8503, 8504, 8505 and superimposed images in which the corresponding CT images 8401, 8402, 8403, 8404, 8405 are superimposed on the material decomposition images may be switched and displayed. Even in these cases, the operator can easily compare these images.

Further, as images for follow-up observation, a plurality of CT images obtained at different times and a plurality of corresponding material decomposition images may be displayed side by side. In addition, a plurality of CT images or a plurality of material decomposition images obtained at different times and a plurality of superimposed images in which one of them is superimposed on the other may be displayed side by side. Even in these cases, the operator can easily compare these images. In this case, a plurality of kinds of images may be displayed so that the CT images and the material decomposition images corresponding to each other, or the CT images or the material decomposition images and their superimposed images corresponding to each other are adjacent to each other. In this case, the operator can observe the CT images and the material decomposition images in a manner that makes it easier to compare them.

In addition, even in a case where the list of thumbnails of each tomographic image included in the three-dimensional CT image is displayed or tomographic images of successive positions are displayed (which is called image flipping), the display controlling unit 304 may cause the CT image and the material decomposition image to be displayed side by side, switched, or superimposed. In this case, the display controlling unit 304 may display a button or a slider for continuously switching and displaying the tomographic images on the display screen. For example, in a case where the CT image and material decomposition image are displayed side by side, the display controlling unit 304 may collectively switch the CT image and the material decomposition image to images at the position corresponding to the operation of the button or the slider and display them. In addition, even in a display of time-lapse difference in which difference of a plurality of images obtained at different times and the reference image are displayed side by side, the difference and the reference image of the CT image and the difference and the reference image of the material decomposition image may be juxtaposed or switched and displayed, or superimposed images of those images may be displayed.

Also, in a case where a plurality of CT images obtained at different times are successively switched and displayed, these images and the corresponding material decomposition images may be juxtaposed or switched and displayed, or superimposed images of those images may be displayed. In this case, if the CT image and the material decomposition image are switched and displayed, the CT image and the material decomposition image can be switched and displayed for subsequential images according to the timing of operation of the switch button.

If the display control process by the display controlling unit 304 is completed in step S304, a series of processes according to the Embodiment 1 ends.

As described above, the image processing apparatus 30 according to the Embodiment 1 includes an obtaining unit 301 and a display controlling unit 304. The obtaining unit 301 obtains an intensity image of radiation, which is obtained by imaging a subject using radiation, and a material decomposition image, which is an image obtained by imaging the subject by counting photons of radiation and indicates a discriminated material. The display controlling unit 304 causes the intensity image of radiation and the material decomposition image to be juxtaposed, switched, or superimposed and displayed on the display unit 307.

With this configuration, the operator can easily compare the CT image and the material decomposition image. Therefore, the operator can easily grasp the relationship between a tissue containing a target material and a tissue not containing the target material, and can perform the observation more efficiently than when observing the material decomposition image alone.

In addition, the display controlling unit 304 may cause the display unit 307 to display options for selecting a material to be discriminated around the material decomposition image. In this case, the operator can easily compare the intensity image of radiation with the material decomposition image while appropriately switching the material decomposition image according to the purpose of observation, and can perform the observation more efficiently.

The intensity image of radiation and the material decomposition image may be generated using common data obtained by imaging the subject using the radiation. In the Embodiment 1, as described above, projection data are obtained from data obtained by imaging using a gantry apparatus 10, and the CT image and the material decomposition image are generated based on the projection data. In this case, since the intensity image of radiation and the material decomposition image show tissues having a common shape, etc., the intensity image and the material decomposition image of radiation can be more easily compared.

However, the intensity image of radiation and the material decomposition image which the display controlling unit 304 causes the display unit 307 to display may be displayed for the comparison for the purpose of easily grasping the relationship between tissues, and they do not have to be generated using common data. Therefore, the intensity image of radiation and the material decomposition image which the display controlling unit 304 causes the display unit 307 to display may be generated using different data. For example, the intensity image of radiation may be generated using data obtained by a CT system that does not use the photon counting technology, and the material decomposition image may be generated using data obtained by a CT system that uses the photon counting technology.

In addition, the material decomposition image displayed on the display unit 307 may include an image obtained by superimposing a plurality of material decomposition images that discriminates different kinds of materials. In this case, it is easy for the operator to grasp the relationship between the tissues containing different kinds of materials and the tissues that do not contain those materials, and it is possible to perform the observation more efficiently.

In addition, the material decomposition image displayed on the display unit 307 may include a plurality of material decomposition images that discriminate different kinds of materials. In this case, since a plurality of material decomposition images relating to a plurality of kinds of materials are displayed, the operator can easily compare the tissues containing the materials with respect to the different kinds of materials, and it is possible to perform the observation more efficiently.

The display controlling unit 304 may cause a plurality of material decomposition images to be arranged adjacent to the intensity image of radiation and displayed them on the display unit 307. In this case, the operator can easily compare each material decomposition image with the intensity image of radiation, and it is possible to perform the observation more efficiently.

In addition, the display controlling unit 304 may cause a plurality of the intensity images of radiation and a plurality of the material decomposition images to be switched collectively and displayed on the display unit 307. The display controlling unit 304 may cause the plurality of intensity image of radiation or the plurality of material decomposition images and a plurality of superimposed images of the plurality of intensity image of radiation and the plurality of material decomposition images to be switched collectively and displayed on the display unit 307. In these cases, the operator can observe the plurality of intensity image of radiation and the plurality of material decomposition images in a manner that is easy to compare with each other by simpler operation, and can perform the observation more efficiently.

Furthermore, the display controlling unit 304 may set transparency for one of the intensity image of radiation and the material decomposition image in response to an instruction of an operator, and cause the one to be superimposed on the other of the intensity image of radiation and the material decomposition image and displayed on the display unit 307. In this case, the operator can check the superimposed image which is obtained by superimposition according to the desired transparency for the intensity image and the material decomposition image of radiation. Therefore, the operator can observe the superimposed images in a manner that is easy for the operator to observe, and can perform the observation more efficiently.

Furthermore, in a case where the intensity image of radiation and the material decomposition image which are juxtaposed and displayed are switched to an intensity image of radiation and a material decomposition image for a different position of the subject, the display controlling unit 304 may cause the intensity image of radiation and the material decomposition image to be switched collectively and displayed on the display unit 307 in response to an instruction of the operator. In this case, the operator can observe the intensity image of radiation and the material decomposition image with respect to a cross section at the desired position by simple operation, and can perform the observation more efficiently.

Furthermore, the image processing apparatus 30 may further include an analyzing unit 303 for analyzing at least one of the intensity image of radiation and the material decomposition image. In this case, the display controlling unit 304 may cause the analysis result by the analyzing unit 303 to be arranged around the analyzed image or superimposed on one of the intensity image of radiation and the material decomposition image and displayed on the display unit 307. In this case, the operator can easily compare the analysis result obtained by analyzing the image in addition to the intensity image of radiation and the material decomposition image, and can perform the observation more efficiently.

Furthermore, the display controlling unit 304 may cause the display unit 307 to display information on imaging failure of at least one of the intensity image of radiation and the material decomposition image in response to an instruction of the operator. In this case, the operator can easily grasp the presence or absence of imaging failure and the reason thereof when observing the image, and can perform the observation more efficiently.

### (Modification 1)

In the Embodiment 1, the display screen for displaying the CT image representing the intensity image of radiation and the material decomposition image in the side-by-side, switched, or superimposed manner was described. On the other hand, the display controlling unit 304 may select one of these display screens and cause the display unit 307 to display it according to the operator's instruction or the pre-setting.

In this case, the display controlling unit 304 selects a display screen to be displayed according to the operator's instruction or the pre-setting, and causes the display unit 307 to display the selected display screen. For example, if the operator instructs to select a display screen for side-by-side display of the CT image and the material decomposition image, the display controlling unit 304 can cause the display unit 307 to display the display screen 501 shown in FIG. 5 according to the instruction.

In addition, the display controlling unit 304 may switch the display screen to be displayed and cause the display unit 307 to display the switched display screen according to the operator's instruction. For example, if the operator further instructs to select the display screen for displaying the CT image and the material decomposition image by switching them, the display controlling unit 304 may switch the display screen 501 shown in FIG. 5 to the display screen 701 shown in FIG. 7A and cause the display unit 307 to display the display screen 701 according to the instruction.

### (Modification 2)

In addition, the display screen shown in the Embodiment 1 corresponds to an analysis screen in which a detailed analysis result can be displayed and a display screen for image observation. On the other hand, the image processing according to the Embodiment 1 can similarly be applied to displaying a preview image such as a CT image on the confirmation screen for confirming an image imaged for the object to be examined S just after the imaging. In the confirmation screen, the CT image which takes a short processing time may be displayed prior to the material decomposition image and the material decomposition image may be switched and displayed later. In this case, the operator can confirm the CT image which takes the short processing time at an early stage and can determine the success or failure of imaging at an early stage.

The image or the analysis result of the image to be displayed on the confirmation screen may be simpler than the image or the analysis result of the image to be displayed on the analysis screen or display screen described above. That is, the display controlling unit 304 may cause the display unit 307 to display an image or an analysis result of image simpler than the image or the analysis result of the analysis screen for analyzing the details of subject as the image or the analysis result of the image on the confirmation screen to be displayed just after the subject is imaged. For example, an image or an analysis result of the image based on data in which the amount of data is thinned out by omitting predetermined data or the like can be displayed on the confirmation screen. In this case, the processing time until display of the confirmation screen can be shortened, and the operator can determine the success or failure of the imaging at an early stage.

The image processing apparatus 30 can calculate an evaluation value of the captured image when display the confirmation screen. In a case where the calculated evaluation value is equal to or less than a threshold value, the image processing apparatus 30 may determine that re-imaging is necessary and indicate a display for recommending re-imaging on the confirmation screen. An evaluation index of the image may be, for example, a Q-index value, but it is not limited to this, and any known evaluation index such as a SN ratio or a contrast value may be used. Any known method for calculating the Q index value may be used. The display controlling unit 304 may also display the calculated evaluation value of the image on the confirmation screen. In these cases, the operator can more efficiently determine whether re-imaging is necessary.

### (Modification 3)

The display screen shown in the Embodiment 1 is an example. Therefore, display items may be added or omitted according to the desired configuration, and the configuration and arrangement of the display screen may be changed according to the desired configuration. For example, the intensity image of radiation and the material decomposition image may be displayed to arranging them upside and downside. In addition, the kind of the image, the imaging date of the image, the analysis result, the kind of the material, and the like may be superimposed on the corresponding image and display, or may be displayed in the vicinity, such as the upper, lower, left, and right, of the corresponding image. For example, the display controlling unit 304 may display the information indicative of the kind of the image around the intensity image of radiation and the material decomposition image, or superimpose it on the intensity image of radiation and the material decomposition image and display them. Further, for example, imaging information including the dose of radiation, amount and a kind of contrast medium, and an imaged site used for imaging may be displayed around the image, such as in the upside, downside, left side, and right side of the image, or may be superimposed on the image and displayed.

In addition, as described above, the images to be displayed for comparison may be images obtained using different imaging apparatus, and for example, the CT image may be obtained by a CT apparatus that does not use the photon counting technology. For this reason, a kind, a model, or the like of the apparatus used for the image obtainment may be displayed around the image, such as the upside, downside side, left side, and right side, or superimposed on the image and displayed.

In addition, the analysis results may be superimposed on various images and displayed according to the operation of buttons (not shown). In addition, for the analysis results of the CT image and the material decomposition image, the analysis results of one image may be superimposed on the other image and displayed. For example, the analysis result of the material decomposition image may be superimposed on the CT image, or the analysis result of the CT image may be superimposed on the material decomposition image and displayed, depending on the operator's operation.

### (Modification 4)

Note that in the Embodiment 1, the transfer order of the imaged data, the generation order of the images, and the display order of the images are not particularly limited. On the other hand, the order may be determined by the operator's instruction or the pre-setting. As described above, the pre-setting may include at least one of the setting preset for each imaging-condition including the imaged site and the like and setting preset for each imaging-mode according to disease and the like.

For example, for data transferred from the DAS 18 of the gantry apparatus 10 to the image processing apparatus 30, the transfer order of data may be determined for each energy band of the radiation. Depending on the purpose of observation, the display of an image based on data in high-energy band or an image based on data in low-energy band may be desired. Therefore, for data transferred from the DAS 18 to the image processing apparatus 30, the transfer order of data may be determined for each energy band in accordance with a pre-setting and the purpose of observation. In this case, according to the pre-setting or the purpose of observation, the obtaining unit 301 may obtain data on a specific energy band obtained by imaging the subject earlier than data on other energy bands. Therefore, the operator can check the desired image more efficiently.

In addition, regarding the image to be generated for display, the generation order of the images may be determined so as to preferentially generate an image of a cross section, which is desired to be checked and displayed first. When observing the CT image or the material decomposition image, it may be desired to preferentially check a cross section image of a predetermined position or a cross section image of a predetermined tissue according to the purpose of observation. Therefore, the generating unit 302 may determine the generation order of the images according to the pre-setting and the purpose of observation. Specifically, the generating unit 302 can generate an intensity image of radiation and a material decomposition image of a specific position of the subject earlier than an intensity image of radiation and a material decomposition image of other positions in the subject according to the pre-setting and the purpose of observation. A cross section to be preferentially generated may be determined according to the pre-setting or the purpose of observation, or may be automatically determined using the result of material decomposition performed prior to the image generation.

Regarding the display order of the images, the display controlling unit 304 may cause the display unit 307 to display the intensity image of radiation such as a CT image and then display the material decomposition image according to the operator's instruction or the pre-setting. For example, the display controlling unit 304 can cause the display unit 307 to display an intensity image of radiation after intensity image of radiation which takes a relatively short processing time is generated, and then display a material decomposition image after a material decomposition image which takes a relatively long processing time is generated. In such a case, the intensity image of radiation and the material decomposition image may be images generated using common data obtained by imaging the subject using the radiation.

However, for the display order, it may be desired to perform the display of the intensity image of radiation and the display of the material decomposition image at the same time. Therefore, depending on the instruction of the operator, it may be possible to choose to display the material decomposition image after the intensity image of radiation, or to display the intensity image and the material decomposition image of radiation at the same time. In these cases, the purpose of observation may be determined based on the operator's instruction regarding the imaging-condition such as the imaged site, or the operator's instruction regarding the purpose of observation. Moreover, the above-described Modifications 1 to 4 are also applicable to the following embodiment.

### Embodiment 2

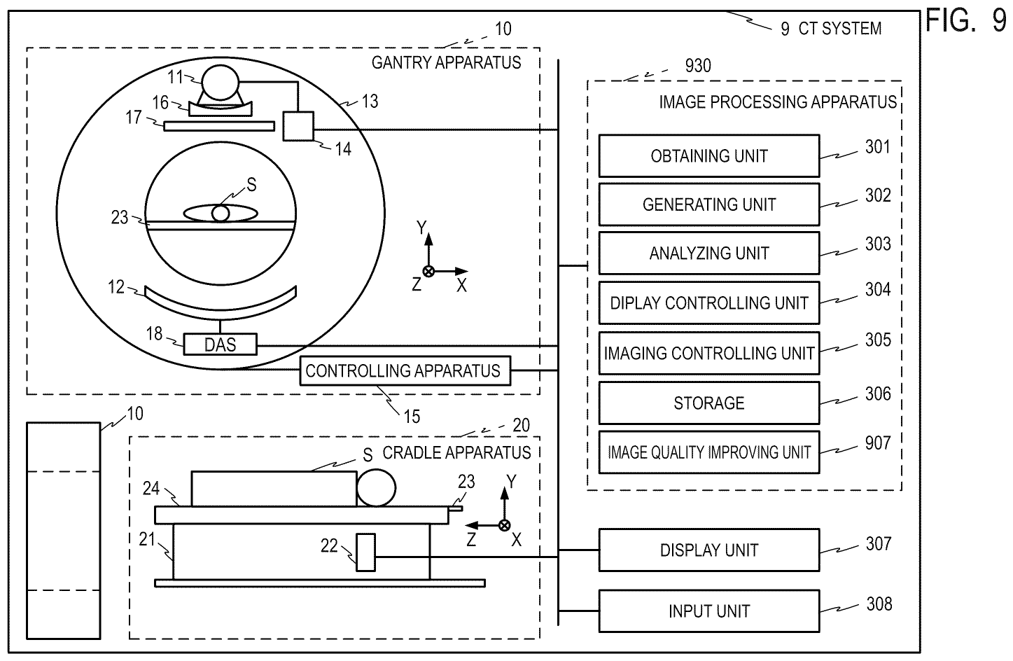

The Embodiment 2 of the present disclosure describes an example of performing image quality improvement processing to improve image quality for an intensity image of radiation and a material decomposition image using a machine learning model that has been trained. Hereinafter, with reference to FIG. 9 to FIG. 12, a CT system 9 according to the Embodiment 2 will be described focusing on the differences with the CT system 1 according to the Embodiment 1. Among the configuration of the CT system 9 according to the Embodiment 2, configuration similar to the configuration of the CT system 1 according to the Embodiment 1 will be denoted by the same reference numerals and will not be described in detail.

In the following, the term “machine learning model” means a learning model based on the machine learning algorithm. Specific algorithms of the machine learning include the nearest neighbor method, the naive Bayes method, the decision tree, the support vector machine, etc. There is also the deep learning, which generates the characteristic amount and the combining weighting factors for learning by itself using the neural network. There is also a method using the gradient boosting such as the LightGBM and the XGBoost as an algorithm using the decision tree. As appropriate, one of the above algorithms can be applied to the following example and modifications. Teacher data refers to training data, which consists of a pair of input data and output data (ground truth).

The term “learned model” refers to a model in which a machine learning model according to any machine learning algorithm such as the deep learning is trained (learned) using appropriate teacher data (raining data) in advance. However, although a learned model has been obtained using an appropriate training data in advance, the learned model is not a model that does not perform further training, and incremental training may be performed. The incremental learning can be performed even after the device is installed at the place of use.

FIG. 9 schematically shows an example of the configuration of the CT system 9 according to the Embodiment 2. The image processing apparatus 930 in the CT system 9 according to the Embodiment 2 includes an image quality improving unit 907 in addition to the components of the image processing apparatus 30 according to the Embodiment 1. The image quality improving unit 907 uses the CT image and/or the material decomposition image generated by the generating unit 302 as an input to the learned model to obtain a CT image and/or a material decomposition image with the improved image quality.

The analyzing unit 303 according to the Embodiment 2 can perform analysis processing on the high-image-quality CT image and/or the high-image-quality material decomposition image obtained by the image quality improving unit 907. The display controlling unit 304 according to the Embodiment 2 can cause the display unit 307 to display the high-image-quality CT image, the high-image-quality material decomposition image, and the like obtained by the image quality improving unit 907.

### (Image Quality Improving Model)

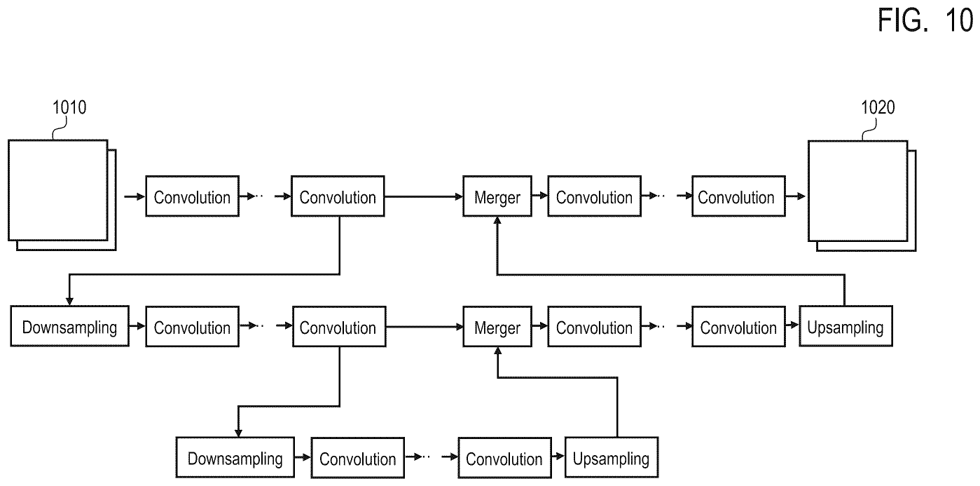

The learned model (image quality improving model) for improving the image quality according to the Embodiment 2 will be described below with reference to FIG. 10. In the Embodiment 2, the image quality improving model is stored in the storage 306 and used by the image quality improving unit 907 for the image quality improvement processing, but the image quality improving model may be provided in an external device (not shown) connected to the image processing apparatus 930.

The image quality improving model in the Embodiment 2 is learned model obtained by performing the training (learning) according to the machine learning algorithm. In the Embodiment 2, for training the machine learning model according to the machine learning algorithm, training data including a pair group of input data which is a low quality image having a specific imaging-condition assumed as a processing object and output data which is a high-image-quality image corresponding to the input data is used. The specific imaging-condition includes, specifically, a predetermined imaged site, imaging system, tube voltage of X-ray, image size, and the like.

The image quality improving model according to the Embodiment 2 is configured as a module which outputs a high-image-quality image based on an input low-image-quality image. Here, the term “image quality improvement” in this specification means to generate an image with a quality suitable for the image inspection from an input image, and the term “high-image-quality image” means the image with the quality suitable for the image inspection. The term “low-image-quality image” refers to, for example, a two-dimensional image or three-dimensional image obtained by CT or the like, or a three-dimensional moving image of CT taken continuously, etc, and is an image which has been obtained without particularly setting to generate a high-image-quality image. Specifically, the low-image-quality image includes, for example, an image taken with a low dose by the CT or the like.

If the high-image-quality image with little noise or high contrast is used for various analysis processing or image analysis such as the region segmentation processing of an image such as the CT image, the analysis can often be performed with higher accuracy than using low-image-quality image. Therefore, the high-image-quality image output by the image quality improving engine (image quality improving model) may be useful not only for the image inspection but also for the image analysis.

Also, the content of the image quality suitable for the image inspection depends on what is desired to be inspected by various image inspections. Therefore, it cannot be said generally, but for example, the image quality suitable for the image inspection includes the image quality with little noise, high contrast, imaging-target in colors and gradations that are easy to observe, large image size, and/or high resolution. Also, the image quality may include image quality in which objects or gradients that do not actually exist and are drawn during the image generation process are removed from the image.

In the image quality improvement processing executed by the image quality improving unit 907 in the Embodiment 2, processing using various machine learning algorithms such as deep learning is performed. In the image quality improvement processing, in addition to the processing using the machine learning algorithm, any existing processing such as various image filter processing, matching processing using a database of high-image-quality images corresponding to similar images, and knowledge-based image processing may be performed.

A configuration example of a CNN (Convolutional Neural Network) related to the image quality improving model according to the Embodiment 2 will be described below with reference to FIG. 10. FIG. 10 shows an example of the configuration of the image quality improving model. The configuration shown in FIG. 10 is composed of a plurality of layer groups responsible for processing a group of input values and outputting the processed values. As shown in FIG. 10, the kinds of the layers included in the configuration includes a convolution layer, a downsampling layer, an upsampling layer, and a merging layer (merger).

The convolution layer performs convolution processing on the group of input values according to parameters such as the kernel size of the filter, the number of filters, the stride value, and the dilation value. The number of dimensions of the kernel size of the filter may also be changed according to the number of dimensions of the input image.

The downsampling layer performs processing to reduce the number of output values to less than the number of input values by thinning or combining input values. Specifically, such processing includes, for example, Max Pooling processing.

The upsampling layer performs processing to increase the number of output values to more than the number of input values by duplicating input values or adding values interpolated from input values. Specifically, such processing includes, for example, linear interpolation processing.

The merging layer is a layer to which values, such as the output values of a certain layer and the pixel values constituting an image, are input from a plurality of sources, and that combines them by concatenating or adding them.

In such a configuration, the values obtained by outputting pixel values constituting the input image 1010 via the convolutional processing block, and pixel values constituting the input image 1010 are combined in merging layer. Then, the high-image-quality image 1020 is generated using the combined pixel values in the last convolutional layer.

Note that caution is required, since when the setting of the parameters to the layers and nodes constituting a neural network is different, the degree with respect to tendency trained from the training data, that can be reproduced at the inference may be different. In other words, in many cases, since appropriate parameters are different depending on the mode at the time of implementation, the parameters can be changed to preferable values according to the needs.

Additionally, the CNN may obtain better characteristics not only by changing the parameters as described above, but also by changing the configuration of the CNN. The better characteristics are, for example, a high accuracy of the noise reduction on a radiation image which is output, a short time for processing, and a short time taken for training of a machine learning model.

Note that the configuration of the CNN used in the Embodiment 2 is a U-net type machine learning model that includes the function of an encoder including a plurality of hierarchies including a plurality of downsampling layers, and the function of a decoder including a plurality of hierarchies including a plurality of upsampling layers. In other words, the configuration of the CNN includes a U-shaped configuration that has an encoder function and a decoder function. The U-net type machine learning model is configured (for example, by using a skip connection) such that the geometry information (space information) that is made ambiguous in the plurality of hierarchies configured as the encoder can be used in a hierarchy of the same dimension (mutually corresponding hierarchy) in the plurality of hierarchies configured as the decoder.

Although not illustrated, as an example of change of the configuration of the CNN, for example, a batch normalization (Batch Normalization) layer, and an activation layer using a normalized linear function (Rectifier Linear Unit) may be incorporated after the convolutional layer.

Here, a GPU can perform efficient arithmetic operations by performing parallel processing of larger amounts of data. Therefore, in a case where training is performed a plurality of time using a machine learning algorithm such as deep learning, it is effective to perform the processing with a GPU. Thus, in the Embodiment 2, a GPU is used in addition to the CPU for processing performed by the image quality improving unit 907, which functions as an example of a training unit. Specifically, when a training program including a learning model is executed, the training is performed by the CPU and the GPU cooperating to perform arithmetic operations. Note that, with respect to the processing of the training unit, the arithmetic operations may be performed only by the CPU or the GPU. Further, the image quality improvement processing according to the Embodiment 2 may also be performed by using the GPU, similarly to the training unit. If the learned model is provided in an external apparatus, the image quality improving unit 907 need not function as a training unit.

The training unit may also include an error detecting unit and an updating unit (not illustrated). The error detecting unit obtains an error between output data output from the output layer of the neural network according to input data input to the input layer, and the ground truth. The error detecting unit may calculate the error between the output data from the neural network and the ground truth using a loss function. Further, based on the error obtained by the error detecting unit, the updating unit updates combining weighting factors between nodes of the neural network or the like so that the error becomes small. The updating unit updates the combining weighting factors or the like using, for example, the error back-propagation method. The error back-propagation method is a method that adjusts combining weighting factors between the nodes of each neural network or the like so that the above error becomes small.

Note that, when using some image processing techniques such as image processing using a CNN, it is necessary to pay attention to the image size. Specifically, it should be kept in mind that, to overcome a problem such as the image-quality of a peripheral part of a high-image-quality image not being sufficiently improved, in some cases different image sizes are required for a low image-quality image that is input and a high-image-quality image that is output.

Although it is not specifically described in the Embodiment 2 in order to provide a clear description, in a case where an image-quality improving model is adopted that requires different image sizes for an image that is input to the image-quality improving model and an image that is output therefrom, it is assumed that the image sizes are adjusted in an appropriate manner. Specifically, padding is performed with respect to an input image such as an image that is used in training data for training a machine learning model or an image to be input to an image-quality improving model, or imaging regions at the periphery of the relevant input image are joined together to thereby adjust the image size. Note that, a region which is subjected to padding is filled using a fixed pixel value, or is filled using a neighboring pixel value, or is mirror-padded, in accordance with the characteristics of the image-quality improving technique so that image-quality improving can be effectively performed.

Further, an image-quality improving processing in the image quality improving unit 907 may be performed using only one image processing technique, and be performed using a combination of two or more image processing techniques. In addition, processing of a group of a plurality of image-quality improving techniques may be performed in parallel to generate a plurality of high-image-quality images, and a high-image-quality image with the highest image-quality may be then finally selected as the high-image-quality image. Note that, the selection of the high-image-quality image with the highest image-quality may be automatically performed using image-quality evaluation indexes, or may be performed by displaying the plurality of high-image-quality images on a user interface (UI) provided in the display unit 307 or the like so that selection may be performed according to an instruction of the examiner (operator).

Note that, since there are also cases where an image that has not been subjected to image-quality improvement is suitable for image examination, an image that has not been subject to image-quality improvement may be added to the objects for selection of the final image. Further, parameters may be input into the image-quality improving model together with the low-image-quality image. For example, a parameter specifying the degree to which to perform image-quality improving, or a parameter specifying an image filter size to be used in an image processing technique may be input to the image-quality improving model together with the input image.

### (Training Data of Image Quality Improving Model)

Next, training data of the image-quality improving model according to the Embodiment 2 will be described. The input data of the training data according to the Embodiment 2 is a low-image-quality image that is obtained by using the same model of equipment as the gantry apparatus 10 and the same settings as the gantry apparatus 10. Further, the output data (ground truth) of the training data of the image-quality improving model is a high-image-quality image that is obtained by using image processing, such as, averaging processing. Specifically, the ground truth may include, for example, a high-image-quality image obtained by performing the image processing such as the averaging processing on an image group obtained by performing the imaging a plurality of times. Further, the ground truth of the training data may be, for example, a high-image-quality image calculated from a high-image-quality image obtained by the imaging with a dose higher than a dose related to the input data.

By using the image-quality improving model that has been trained in this way, the image quality improving unit 907 can output a high-image-quality image in which noise reduction and the like are performed by the averaging processing and the like. Therefore, the image quality improving unit 907 can generate a high-image-quality image suitable for image inspection based on low-image-quality image, which is an input image.

The example of using averaged image as the output data of the training data is described. However, the output data of the training data of the image-quality improving model is not limited to this example. The ground truth of the training data may be a high-image-quality image corresponding to the input data. Therefore, the ground truth of the training data may be, for example, an image which has been subjected to contrast correction suitable for inspection, an image of which the resolution has been improved, etc. Further, an image obtained from an image obtained by performing image processing using statistical processing such as maximum a posteriori probability estimation (MAP estimation) processing on a low-image-quality image as the input data may be used as the ground truth of the training data. Any known method may be used for generating the high-image-quality image.

In addition, a plurality of image-quality improving models independently performing various image quality improving processing such as noise reduction, contrast adjustment, and resolution improvement may be prepared as the image quality improving model. Further, one image quality improving model performing at least two image quality improving processing may be prepared. In these cases, a high-image-quality image corresponding to the desired processing may be used as the ground truth of the training data. For example, for an image quality improving model that includes individual processing such as noise reduction processing, a high-image-quality image that has be subjected to the individual processing such as the noise reduction processing may be used as the output data of the training data. For an image quality improving model for performing a plurality of image quality improvement processing, for example, a high-image-quality image that has been subjected to noise reduction processing and contrast correction processing may be used as the ground truth of the training data.

By using such training data, it is possible to construct a learned model which generates a high-image-quality image with improved image quality. According to such a learned model, for example, since high-image-quality image can be obtained by using a low-image-quality image photographed at a low dose as an input, the amount of radiation used for photographing can be reduced, and it can be expected that the occurrence of pile-up or the like in photon counting technology can be suppressed.

Note that the learned model may be prepared for each kind of images. For example, by performing training using training data composed of a pair of a low-image-quality CT image and a high-image-quality CT image, it is possible to obtain a learned model which outputs a high-image-quality CT image using a low-image-quality CT image as an input. Similarly, by performing training using training data composed of a pair of a low-image-quality material decomposition image and a high-image-quality material decomposition image, it is possible to obtain a learned model which outputs a high-image-quality material decomposition image using a low-image-quality material decomposition image as input.

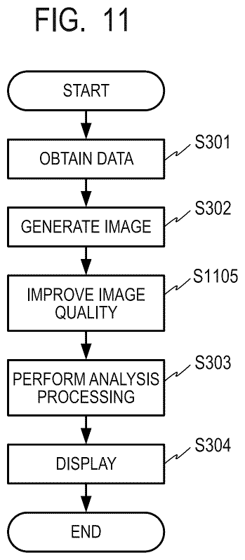

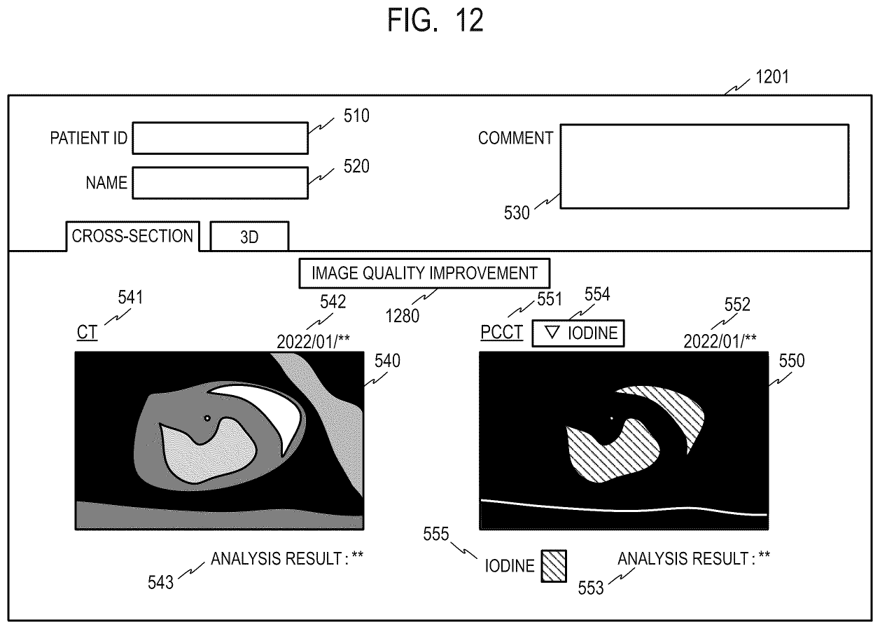

Next, a series of processes including the image processing according to the Embodiment 2 will be described with reference to FIG. 11 and FIG. 12. FIG. 11 shows a flowchart of a series of processes according to the Embodiment 2. Among process shown in FIG. 11, processes that are similar to the series of processes according to the Embodiment 1 shown in FIG. 3 is denoted using the same reference numerals as the process according to the Embodiment 1, and a detailed description thereof will be omitted. If the process is started and steps S301 and S302 are performed, the process proceeds to step S1105. Note that, in step S301, the obtaining unit 301 can obtain data obtained by imaging the object to be examined S at a low dose, for example.

In step S1105, the image quality improving unit 907 obtains a high-image-quality CT image and/or a high-image-quality material decomposition image by using a CT image and/or a material decomposition image generated by the generating unit 302 as an input to the image quality improving model. The image quality improving model may be prepared for each kind of images or each kind of materials, and the image quality improving unit 907 can select and use an image quality improving model to be used for the image quality improving process according to a kind of image to be subjected to the image quality improvement or a kind of material to be discriminated. A plurality of image quality improving models may be provided according to the imaging-condition such as a dose or an imaged site. In this case, the image quality improving unit 907 can perform the image quality improvement processing using the image quality improving model corresponding to the imaging-condition of the image to be used as the input. The learned model corresponding to a kind of image, a kind of material, and the imaging-condition can be obtained by training using training data of each kind of the images, training data of each kind of the materials, and training data of each imaging-condition.

In step S303, the analyzing unit 303 analyzes the high-image-quality CT image and/or the material decomposition image obtained by the image quality improving unit 907. The analysis processing may be the same as the analysis processing performed in step S303 according to the Embodiment 1. Note that, similarly to the Embodiment 1, the analyzing unit 303 can also analyze the CT image and/or material decomposition image before the image quality is improved.

In step S304, the display controlling unit 304 causes the high-image-quality CT image and the high-image-quality material decomposition image obtained by the image quality improving unit 907 to be juxtapose, switched, or superimposed and displayed on the display unit 307. In addition, the display controlling unit 304 causes the display unit 307 to display the analysis results of the analyzing unit 303 using high-image-quality image. The display screen display on the display unit 307 by the display controlling unit 304 may be the same as the display screen described in the Embodiment 1, and the high-image-quality CT image and the material decomposition image can be displayed as the CT image and the material decomposition image to be displayed.

In the display screen display by the display controlling unit 304, it is also possible to switch and display the images before and after the image quality improvement. FIG. 12 shows an example of a display screen 1201 according to the Embodiment 2. The display screen 1201 is a display screen similar to the display screen 501 shown in FIG. 5, but the display screen 1201 further includes an image quality improvement button 1280. When the image quality improvement button 1280 is operated by the operator via the input unit 308, the display controlling unit 304 switches the CT image 540 and the material decomposition image 550 to the CT image and material decomposition image collectively after the image quality improvement. Note that the image quality improvement button 1280 may be provided for each image to be subjected the image quality improvement.

This process can similarly be applied to the other display screens 601, 701, 702, 703, 801, 802 and the like described in the Embodiment 1. In addition, it can similarly be applied to the thumbnail list and the time-lapse difference described in the Embodiment 1.

Further, when the display controlling unit 304 switches between the images before and the image after the image quality improvement and causes them to be displayed, the display controlling unit 304 may switch the displayed analysis result to the analysis result corresponding to the image to be switched and displayed and cause them to be displayed. For example, when the image quality improvement button 1280 is operated to switch the CT image 540 and the material decomposition image 550 to the images after the image quality improvement, the display controlling unit 304 can switch the analysis result 543, 553 to the analysis result of the images after the image quality improvement to collectively. In a case where the image quality improvement button is provided for each image, the display controlling unit 304 can switch the analysis result corresponding to the image to be switched between the analysis result of the image before image quality improvement and the analysis result of the image after the image quality improvement. In step S304, if the display control process by the display controlling unit 304 is completed, the series of processes according to the Embodiment 2 ends.

As described above, the display controlling unit 304 according to the Embodiment causes an image with higher image quality than the at least one of an intensity image of radiation and a material decomposition image, which is obtained by using the at least one as an input to a learned model, to be switched from the at least one image and displayed on the display unit 307 in response to an instruction of the operator. In this case, the operator can appropriately observe the intensity image of radiation and the material decomposition image of which the image quality is improved in a state in which it is easy to compare them, and can perform the observation more efficiently.

The learned model may include at least one of a plurality of learned models according to the kind of the image and a plurality of learned models according to the kind of the material to be discriminated. In this case, it is possible to obtain an image that is more appropriately subjected to the image quality improvement processing according to a kind of the image and a kind of the material to be discriminated by the learned model, and the operator can observe an image with the higher image quality more efficiently.

In the Embodiment 2, an example of using the image quality improving model for performing the image quality improvement processing has been described. However, processing using a learned model is not limited to the image quality improvement processing, and may be the segmentation processing or the like. In this case, as training data of the learned model, a CT image or a material decomposition image may be used as input data, and a label image which is labeled by a physician or the like for each region on the input data may be used as output data (ground truth). The output data may be a label image labeled by well-known rule-based segmentation processing. The rule-based processing refers to processing based on the regularity of organization or the like. The configuration of a machine learning model may be similar to that of image quality improving model described above.

In this case, by using at least one image of the intensity image of radiation or the material decomposition image generated by the generating unit 302 as an input to the learned model, the image processing apparatus 930 can obtain an image in which each region in the at least one image used as an input is labeled. The learned model may also be provided for each kind of the images and each kind of the materials. In this case, the image processing apparatus 930 can select and use a learned model to be used for the segmentation processing according to the kind of the image to be segmented and the kind of the material to be discriminated. Further, a plurality of learned models may also be provided according to the imaging-condition such as a dose or an imaged site. In this case, the image processing apparatus 930 can perform the segmentation processing using a learned model corresponding to the imaging-condition of the image to be used as an input. Note that the learned models corresponding to the kinds of the images, the kinds of materials, or the imaging-condition can be obtained by training using training data for each kind of the images, training data for each kind of the materials, or training data for each imaging-condition.

The display controlling unit 304 can cause the obtained label image to be displayed on the display screen as an analysis result. Note that for the display of the analysis result using the learned model, a corresponding switching button may be provided, and the display controlling unit 304 may control ON/OFF or the like of the analysis result according to the operation of the switching button by the operator. The display controlling unit 304 may switch the obtained label image from at least one of the corresponding intensity image of radiation or the corresponding material decomposition image and cause it to be displayed on the display unit 307.

Also, the display controlling unit 304 may change the presence or absence of display of an image quality improvement button or a switching button for accepting the instruction of the operator regarding the processing using a learned model for segmentation, according to the imaging-condition of the image. For example, the display controlling unit 304 may not cause the image quality improvement button or the switching button for segmentation to be displayed on the display unit 307 in a case where an image obtained by imaging-condition that does not match with imaging-condition of the training data of the prepared learned model is display. On the other hand, the display controlling unit 304 may cause the image quality improvement button or the switching button for segmentation to be displayed on the display unit 307 in a case where an image obtained by imaging-condition that matches with imaging-condition of training data of the prepared learned model.

Here, the imaging-condition may include the kind of the image. In this case, for example, in a case where a learned model for CT image is not prepared, the display controlling unit 304 may not cause the switching button for processing using the learned model for CT image to be displayed on the display screen for displaying the CT image.

Note that the image quality improving unit 907 or the image processing apparatus 930 may determine whether or not the processing can be performed based on the imaging-condition if the image quality improvement processing or the segmentation processing using learned model is performed. In this case, the display controlling unit 304 may change whether or not to display the image quality improvement button or the switching button for segmentation according to the determination result by the image quality improving unit 907 or the image processing apparatus 930. For example, if the image quality improving unit 907 determines that the image quality improvement processing cannot be performed based on imaging-condition, the display controlling unit 304 may not cause the image quality improvement button to be displayed on the display screen based on the determination by the image quality improving unit 907.

In these cases, if the processing using the learned model cannot be performed, the buttons related to the processing are not displayed on the display screen, so that it is possible to prevent an operator from performing an inappropriate operation. In a case where the processing using the learned model cannot be performed, the display controlling unit 304 may cause a message indicating that the processing cannot be performed to be displayed on the display screen.

The image quality improvement processing or the segmentation processing using the learned model may be performed according to the operation of the corresponding image quality improvement button or the corresponding switching button. In this case, if the processing using the learned model cannot be performed based on the imaging-condition or the like, the display controlling unit 304 may cause a message indicating that the processing cannot be performed to be displayed on the display screen.

Note that in a case where an image is obtained using a machine learning model, an image showing a tissue or the like that does not actually exist may be generated. Therefore, in a case where a high-image-quality image or a label image obtained using the learned model is displayed, the display controlling unit 304 may cause a message indicating that the image was obtained using a machine learning model. In this case, the operator observes the image while recognizing that the image was obtained using the machine learning model, so that erroneous judgment or the like caused by the processing using the machine learning model can be prevented.

It should be noted that the training data of the image quality improving model and the learned model for segmentation is not limited to data obtained using the actual imaging apparatus. The image data may be data obtained using the same type of imaging apparatus, data obtained using the same kind of imaging apparatus, or the like, depending on the desired configuration.

### (Modification 5)

In the various embodiments and modifications described above, an instruction from the examiner relating to the display, analysis, image quality improvement processing, and segmentation processing may be a voice instruction or the like in addition to a manual instruction (for example, an instruction using a user interface or the like). At such time, for example, a machine learning model including a voice recognition model (a voice recognition engine or a learned model for voice recognition) obtained by machine learning may be used. In addition, a manual instruction may be an instruction by character input using a keyboard, a touch panel, or the like. At such time, for example, a machine learning model including a character recognition model (a character recognition engine or a learned model for character recognition) obtained by machine learning may be used. Further, an instruction from the examiner may be an instruction by a gesture or the like. At such time, a machine learning model including a gesture recognition model (a gesture recognition engine or a learned model for gesture recognition) obtained by machine learning may be used.

Further, an instruction from the examiner may be a result of detection of the line of sight of the examiner on the monitor. The line-of-sight detection result may be, for example, a pupil detection result using a moving image of the examiner obtained by imaging from around the monitor. At such time, the pupil detection from the moving image may use an object recognition engine as described above. Further, an instruction from the examiner may be an instruction by brain waves, or a faint electric signal flowing through the body or the like.

In such a case, for example, the training data may be training data in which character data or voice data (waveform data) or the like indicating an instruction to display the intensity image of radiation, the corresponding material decomposition image, or the superimposed image is adopted as input data, and an execution command for causing the various images to be actually displayed on the display unit 307 is adopted as output data. Further, the training data may be training data in which, for example, character data or voice data or the like indicating an instruction to display a high-image-quality image obtained with the image quality improving model is adopted as input data, and an execution command for displaying the high-image-quality image and an execution command for changing an image quality improvement button to an active state are adopted as output data. Similarly, the training data may be training data in which, for example, character data or voice data or the like indicating an instruction to display a label image obtained with the learned model for segmentation is adopted as input data, and an execution command for displaying the label image and an execution command for changing the corresponding switching button to an active state are adopted as output data. Note that, any kind of training data may be used as long as, for example, the instruction content indicated by the character data or voice data or the like and the execution command content correspond with each other. Further, voice data may be converted to character data using an acoustic model or a language model or the like. Further, processing that reduces noise data superimposed on voice data may be performed using waveform data obtained with a plurality of microphones. Further, a configuration may be adopted so that a selection between an instruction issued by characters or voice or the like and an instruction input using a mouse, a touch panel, or the like can be made according to an instruction from the examiner. In addition, a configuration may be adopted so that a selection can be made to turn instruction by characters or voice or the like on or off according to an instruction from the examiner.

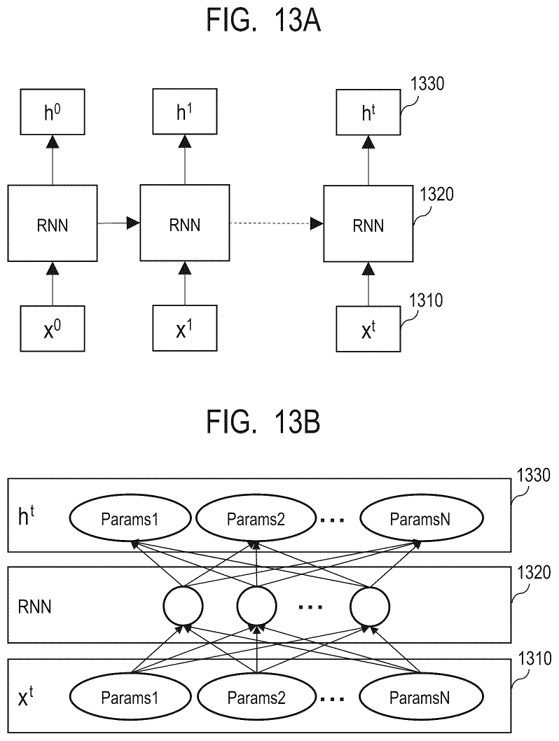

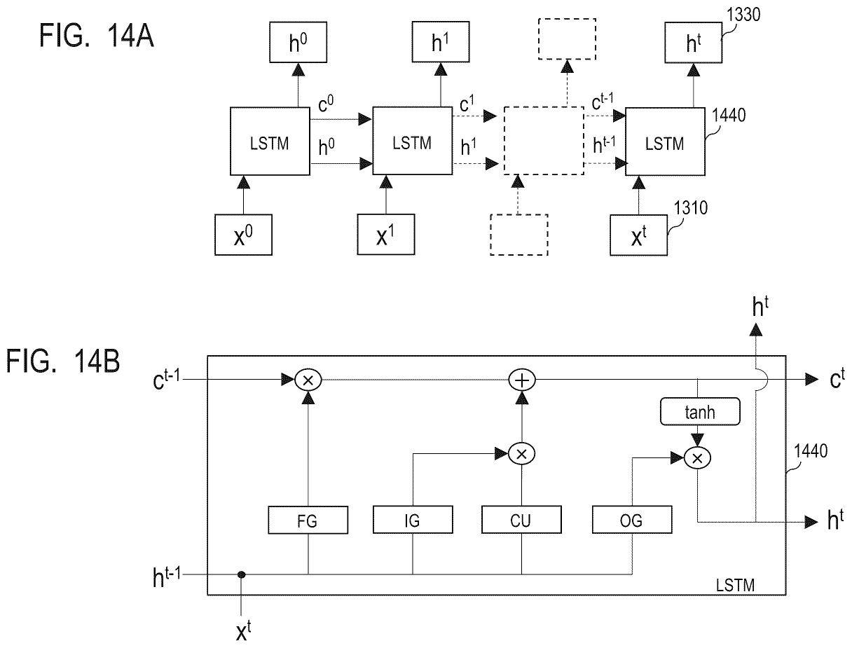

Here, the machine learning includes the deep learning as described above, and a recurrent neural network (RNN), for example, can be used for at least part of the multi-hierarchical neural network. Here, as an example of the machine learning model according to the Modification 5, an RNN that is a neural network that handles time-series information will be described with reference to FIG. 13A and FIG. 13B. Further, a long short-term memory (hereinafter referred to as an “LSTM”), which is a kind of RNN, will be described with reference to FIG. 14A and FIG. 14B.

FIG. 13A illustrates a structure of an RNN that is a machine learning model. An RNN 1320 has a loop structure in the network, data x^{t} 1310 is input at time t, and the RNN 1320 outputs data h^{t} 1330. Since the RNN 1320 has a loop function in the network, the state at the current time can be taken over to the next state, and hence time-series information can be handled. FIG. 13B illustrates an example of the input/output of parameter vectors at time t. Data x^{t} 1310 includes N pieces of data (Params1 to ParamsN). Further, data h^{t} 1330 output from the RNN1320 includes N pieces of data (Params1 to ParamsN) corresponding to the input data.

However, since the RNN cannot handle long-term information during back propagation, the LSTM may be used. The LSTM can learn long-term information by providing a forget gate, an input gate, and an output gate. FIG. 14A illustrates a structure of the LSTM. In an LSTM 1440, information that the network takes over at the next time t is an internal state c^{t-1} of the network called a cell and output data h^{t-1}. Note that lowercase letters (c, h, x) in the figure represent vectors.

Next, the LSTM 1440 is illustrated in detail in FIG. 14B. A forget gate network FG, an input gate network IG, and an output gate network OG are illustrated in FIG. 14B, and each of these is a sigmoid layer. Therefore, a vector in which each element has a value from 0 to 1 is output. The forget gate network FG determines how much past information is held, and the input gate network IG determines which value is to be updated. A cell update candidate network CU is an activation function tanh layer. This creates a vector of new candidate values to be added to the cell. The output gate network OG selects an element of a cell candidate and selects how much information is to be transmitted at the next time.

Note that, the LSTM model described above is a basic form, and the present invention is not limited to the network illustrated here. The coupling between networks may be changed. Further, a QRNN (quasi-recurrent neural network) may be used instead of an LSTM. In addition, the machine learning model is not limited to a neural network, and a machine learning model such as Boosting or Support Vector Machine or the like may be used. In addition, in a case where the instruction from the examiner is input by a character, voice, or the like, a technology relating to the natural language processing (for example, Sequence to Sequence) may be applied. In addition, an interactive engine (interactive model, learned model for interactive) which responds to the examiner by output by a text, voice, or the like may be applied.

Note that, in the image quality improving model and the learned models for segmentations described in the Embodiment 2, it is conceivable for the magnitude of intensity values, and the order and slope, positions, distribution, and continuity of bright sections and dark sections and the like of an image that is input data to be extracted as a part of the feature values and used for inference processing. On the other hand, in the case of the learned models for voice recognition, for character recognition, for gesture recognition and the like, since learning that uses time-series data is performed, it is conceivable to also extract a slope between consecutive time-series data values that are input, as a part of the feature values, and to use the slope for inference processing. Therefore, it is expected that such learned models can be utilized to perform inference with excellent accuracy by using influences caused by changes over time in specific numerical values in inference processing.

Further, the image quality improving model and the learned model for segmentation can be provided in the image processing apparatus 930. These learned models, for example, may be constituted by a software module that is executed by a processor such as a CPU, an MPU, a GPU or an FPGA, or may be constituted by a circuit that serves a specific function such as an ASIC. Further, the learned models may be provided in a different apparatus such as a server that is connected to the image processing apparatus 930. In this case, the image processing apparatus 930 can use the learned models by connecting to the server or the like that includes the learned models through any network such as the Internet. The server that includes the learned models may be, for example, a cloud server, a FOG server, or an edge server. Note that, in a case where a network within the facility, or within premises in which the facility is included, or within an area in which a plurality of facilities are included or the like is configured to enable wireless communication, for example, the reliability of the network may be improved by configuring the network to use radio waves in a dedicated wavelength band allocated to only the facility, the premises, or the area or the like. Further, the network may be constituted by wireless communication that is capable of high speed, large capacity, low delay, and many simultaneous connections.

According to the Embodiments 1-2 and the modifications 1-5 of the present disclosure, the intensity image of radiation and the material decomposition image obtained using photon counting technology can be displayed so that they are easily compared.

In the Embodiments 1 and 2, examples where the generating unit 302 generates the intensity image of radiation such as CT image and the material decomposition image were described. However, the obtaining unit 301 may obtain the intensity image of radiation and the material decomposition image from an image processing apparatus or storage device (not shown) via any network, and the obtained images may be used for the display processing or other image processing.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

The processor or circuit may include a central processing unit (CPU), a micro processing unit (MPU), a graphics processing unit (GPU), an application specific integrated circuit (ASIC), or a field programmable gateway (FPGA). The processor or circuit may also include a digital signal processor (DSP), a data flow processor (DFP), or a neural processing unit (NPU).

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

What is claimed is:

1. An image processing apparatus comprising:
an obtaining unit configured to obtain an intensity image of radiation obtained by imaging a subject using the radiation, and a material decomposition image indicating a discriminated material and obtained by imaging the subject by counting photons of the radiation; and
a display controlling unit configured to cause the intensity image of radiation and the material decomposition image to be juxtaposed, switched, or superimposed and displayed on a display unit.

2. The image processing apparatus according to claim 1, wherein the display controlling unit is configured to causes the display unit to display options for selecting a material to be discriminated around the material decomposition image.

3. The image processing apparatus according to claim 1, wherein the intensity image of radiation and the material decomposition image are images generated using common data obtained by imaging the subject using the radiation.

4. The image processing apparatus according to claim 1, wherein the material decomposition image includes an image obtained by superimposing a plurality of material decomposition images discriminating different kinds of materials on each other.

5. The image processing apparatus according to claim 1, wherein:
the material decomposition image includes a plurality of material decomposition images discriminating different kinds of materials; and
the display controlling unit configured to cause each of the plurality of material decomposition images to be arranged adjacent to the intensity image of radiation and displayed on the display unit.

6. The image processing apparatus according to claim 1, wherein the display controlling unit is configured to:
cause a plurality of the intensity images of radiation and a plurality of the material decomposition images to be switched collectively and displayed on the display unit; or
cause the plurality of intensity image of radiation or the plurality of material decomposition images and a plurality of superimposed images of the plurality of intensity images of radiation and the plurality of material decomposition images to be switched collectively and displayed on the display unit.

7. The image processing apparatus according to claim 1, wherein the display controlling unit is configured to set transparency of one of the intensity image of radiation and the material decomposition image according to an instruction of an operator, and cause the one to be superimposed on the other of the intensity image of radiation and the material decomposition image and displayed on the display unit.

8. The image processing apparatus according to claim 1, wherein:
the display controlling unit is configured to cause the intensity image of radiation and the material decomposition image to be superimposed in a blend ratio of the intensity image of radiation and the material decomposition image in response to an instruction of an operator and displayed on the display unit; and
the blend ratio is a ratio of transparency having a relationship in which one of transparency of the intensity image of radiation and transparency of the material decomposition image is raised and the other is lowered.

9. The image processing apparatus according to claim 1, wherein in a case where the intensity image of radiation and the material decomposition image which are juxtaposed and displayed are switched to an intensity image of radiation and a material decomposition image for a different position of the subject, the display controlling unit is configured to cause the intensity image of radiation and the material decomposition image to be switched collectively and displayed on the display unit in response to an instruction of an operator.

10. The image processing apparatus according to claim 1, wherein the display controlling unit is configured to cause information indicating the kind of an image to be arranged around the intensity image of radiation and the material decomposition image or superimposed on the intensity image of radiation and the material decomposition image and displayed on the display unit.

11. The image processing apparatus according to claim 1, further comprising an analyzing unit configured to analyze at least one of the intensity image of radiation and the material decomposition image, wherein the display controlling unit is configured to:
cause an analysis result by the analyzing unit to be arranged around an analyzed image or superimposed on one of the intensity image of radiation and the material decomposition image and displayed on the display unit; and
cause the display unit to display an analysis result simpler than an analysis result on an analysis screen for analyzing the details of the subject as an analysis result on a confirmation screen to be displayed just after the subject is imaged.

12. The image processing apparatus according to claim 1, wherein the display controlling unit causes the display unit to display information on imaging failure of at least one of the intensity image of radiation and the material decomposition image in response to an instruction of an operator.

13. The image processing apparatus according to claim 1, wherein:
the intensity image of radiation and the material decomposition image are images generated using common data obtained by imaging the subject using the radiation,
the display controlling unit is configured to cause the display unit to display the material decomposition image after the intensity image of radiation is displayed.

14. The image processing apparatus according to claim 1, wherein:
the obtaining unit is configured to obtain data on a specific energy band obtained by imaging the subject earlier than data on other energy bands according to a pre-setting or an instruction of an operator; and
the pre-setting includes at least one of a setting preset for each imaging-condition including an imaged site and a setting preset for each imaging-mode according to disease.

15. The image processing apparatus according to claim 1, further comprising a generating unit configured to generate the intensity image of radiation and the material decomposition image using data obtained by imaging the subject, wherein:
the generating unit is configured to generate an intensity image of radiation and a material decomposition image for a specific position of the subject earlier than an intensity image of radiation and a material decomposition image for other positions of the subject according to a pre-setting or an instruction of an operator; and
the pre-setting includes at least one of a setting preset for each imaging-condition including an imaged site and a setting preset for each imaging-mode according to disease.

16. The image processing apparatus according to claim 1, wherein:
the display controlling unit is configured to:
cause an image with higher image quality than at least one image of the intensity image of radiation and the material decomposition image or an image in which each area of the at least one image is labeled, which is obtained by using the at least one image as an input of a learned model, to be switched from the at least one image and displayed on the display unit in response to an instruction of an operator; and
change the presence or absence of a display for accepting the instruction of the operator based on an imaging-condition of the at least one image, and
the learned model includes at least one of a plurality of learned model according to the kind of an image and a plurality of learned model according to the kind of a material to be discriminated.

17. The image processing apparatus according to claim 1, wherein an instruction of an operator related to display is information obtained by using at least one learned model among a learned model for character recognition, a learned model for voice recognition, and a learned model for gesture recognition.

18. An imaging system comprising:
an imaging apparatus including a radiation detector capable of counting photons of radiation, and
the image processing apparatus according to claim 1.

19. An image processing method comprising:
obtaining an intensity image of radiation obtained by imaging a subject using the radiation, and a material decomposition image indicating a discriminated material and obtained by imaging the subject by counting photons of the radiation; and
causing the intensity image of radiation and the material decomposition image to be juxtaposed, switched, or superimposed and displayed on a display unit.

20. A non-transitory computer-readable storage medium having stored thereon a program for causing, when executed by a computer, the computer to execute respective steps of the image processing method according to claim 19.
